(19) United States
(12) Patent Application Publication
(10) Pub. No.: US 2004/0210035 A1
(43) Pub. Date: Oct. 21, 2004

(54) Survivin-derived peptides and use thereof

(76) Inventors: Eivind Per Thor Straten, Hvidovre (DK); Mads Hald Andersen, Hellerup (DK)

Correspondence Address: HUNTON & WILLIAMS LLP, INTELLECTUAL PROPERTY DEPARTMENT, 1900 K STREET, N.W., SUITE 1200, WASHINGTON, DC, 20006-1109, US

(21) Appl. No.: 10/354,090

(22) Filed: Jan. 30, 2003

Related U.S. Application Data
(60) Provisional application No. 60/352,284, filed on Jan. 30, 2002.

(51) Int. Cl. C07K 14/74

(52) U.S. Cl. 530/350

(57) ABSTRACT

MHC Class I-restricted peptides derived from the tumor associated antigen, survivin, which peptides are capable of binding to Class I HLA molecules at a high affinity, capable of eliciting INF-γ-producing cells in a PBL population of a cancer patient and capable of in situ detection of cytotoxic T cells in a tumor tissue, therapeutic and diagnostic composition comprising the peptide and uses hereof.

### FIELD OF INVENTION

The present invention relates to novel survivin-derived peptides and their use for diagnostic and therapeutic purposes, in particular in cancer. In particular, the novel peptides are MHC Class I-restricted T-cell epitopes that are capable of eliciting cytotoxic T-cell responses in cancer patients including in situ and ex vivo responses. Specifically, such novel peptides are derived from the apoptosis inhibitor protein survivin, a recognized tumor associated antigen (TAA).

### TECHNICAL BACKGROUND AND PRIOR ART

The process by which the mammalian immune system recognizes and reacts to foreign or alien materials is a complex one. An important facet of the system is the T-cell response. This response requires that T cells recognize and interact with complexes of cell surface molecules referred to as human leukocyte antigens (HLA) constituting the human major histocompatibility complex (MHC), and peptides. The peptides are derived from larger molecules, which are processed by the cells, which also present the HLA/MHC molecule. The interaction of T cells and complexes of HLA/peptide is restricted, requiring a T cell that is specific for a particular combination of an HLA molecule and a peptide. If a specific T cell is not present, there is no T-cell response even if its partner complex is present. Similarly, there is no response if the specific complex is absent, but the T cell is present.

The mechanism by which T cells recognize cellular abnormalities has also been implicated in cancer. E.g. in WO92/20356, a family of genes is disclosed which are processed into peptides which, in turn, are expressed on cells surfaces, and can lead to lysis of the tumour cells by specific CTLs. These genes are referred to as the MAGE family and are said to code for “tumour rejection antigen precursors” or “TRAP” molecules, and the peptides derived therefrom are referred to as “tumour rejection antigens” or “TRAs”.

In WO 94/05304, nonapeptides are disclosed which bind to the HLA-A1 molecule. The reference discloses that given the known specificity of particular peptides for particular HLA molecules, one should expect a particular peptide to bind one HLA molecule, but not others. This is significant, because different individuals possess different HLA phenotypes. As a result, while identification of a particular peptide as being a partner for a specific HLA molecule has diagnostic and therapeutic ramifications, these are only relevant for individuals with that particular HLA phenotype.

Thus, it is well established that peptide epitopes derived from tumor associated antigens (TAAs) can be recognized as antigens by cytotoxic T lymphocytes (CTLs) in the context of MHC molecules (1). However, although it is generally accepted that most if not all, tumours are antigenic, only a few are indeed immunogenic in the sense that tumour progression is readily controlled by the immune system.

To overcome this limitation, several immunotherapeutic trials have been initiated, e.g. vaccinations with TAA-derived peptides. For melanoma, the tumour for which the largest number of CTL-defined TAAs has been characterized, powerful CTL responses against antigens have been induced by vaccination and some patients experienced a complete remission of their disease (2,3). However, most of the peptide epitopes used in these vaccination trials are melanocyte specific, and these peptides cannot be applied for tumours of non-melanocyte origin. Furthermore, expression of these TAAs is heterogeneous among tumours from different patients and can even vary among metastases obtained from one patient. However, during the last couple of years a number of tumour specific peptide antigens, which are expressed in a number of different cancers, have been identified, i.e. HER-2 (4), Muc-1 (5) and telomerase (6).

Apoptosis is a genetic program of cellular suicide, and inhibition of apoptosis has been suggested to be an important mechanism involved in cancer formation by extending the life span of cells favouring the accumulation of transforming mutations (7). Survivin is a recently identified member of the family of inhibitors of apoptosis proteins (IAPs). In a global gene expression analysis of about 4 million transcripts, survivin was identified as one of the top genes invariably up-regulated in many types of cancer but not in normal tissue (8). Solid malignancies overexpressing survivin include lung, colon, breast, pancreas, and prostate cancer as well as hematopoietic malignancies (9). Additionally, series of melanoma and non-melanoma skin cancers have been reported to be invariably survivin positive (10,11). The overexpression of survivin in most human cancers suggests a general role of apoptosis inhibition in tumor progression, a notion substantiated by the observation that in the case of colorectal and bladder cancer, as well as neuroblastoma, expression of survivin was associated with an unfavourable prognosis. In contrast, survivin is undetectable in normal adult tissues. These characteristics qualify survivin as a suitable TAA for both diagnostic and therapeutic purposes.

Thus, during the last decade a large number of TAAs have been identified which are recognized by CTLs in a major histocompatibility complex (MHC)-restricted fashion. As survivin is overexpressed in most human cancers and inhibition of its function results in increased apoptosis, this protein may serve as a target for therapeutic CTL responses. The survivin protein and the potential diagnostic and therapeutic use hereof are disclosed in (8) and U.S. Pat. No. 6,245,523, which are incorporated herein by reference. Survivin is a 16.5 kDa cytoplasmic protein containing a single BIR and a highly charged carboxy-terminal coiled coil region instead of a RING finger, which inhibits apoptosis induced by growth factor (IL-3) withdrawal when transferred in B cell precursors. The gene coding for survivin is nearly identical to the sequence of Effector Cell Protease Receptor-1 (EPR-1), but oriented in the opposite direction, thus suggesting the existence of two separate genes duplicated in a head-to-head configuration. Accordingly, survivin can be described as an antisense EPR-1 product. Functionally, inhibition of survivin expression by up-regulating its natural antisense EPR-1 transcript results in massive apoptosis and decreased cell growth.

U.S. Pat. No. 6,245,523 discloses the isolation of purified survivin and it provides nucleic acid molecules that encode the survivin protein, and antibodies and other molecules that bind to survivin. U.S. Pat. No. 6,245,523 also discloses anti-apoptotically active fragments of the survivin protein and variants hereof wherein an amino acid residue has been inserted N- or C-terminal to, or within, the disclosed survivin sequence. It is specifically disclosed that such peptides should contain key functional residues required for apoptosis, i.e. Trp at position 67, Pro at position 73 and Cys at position 84.

The present invention is based on the discovery that MHC Class I restricted peptides can be derived from the survivin protein, which are capable of binding to MHC Class I HLA molecules and thereby eliciting both ex vivo and in situ CTL immune responses in patients suffering from a wide range of cancer diseases. These findings strongly suggest that survivin acts as a TRAP molecule, which is processed by cells into peptides having TRA functionality. Evidently, these findings open the way for novel therapeutic and diagnostic approaches which, due to the fact that survivin appears to be expressed universally by tumour cells, are generally applicable in the control of cancer diseases.

### SUMMARY OF THE INVENTION

Accordingly, the invention pertains in a first aspect to a MHC Class I-restricted epitope peptide derived from survivin, said epitope having at least one of the following characteristics:

(i) capable of binding to the Class I HLA molecule to which it is restricted at an affinity as measured by the amount of the peptide that is capable of half maximal recovery of the Class I HLA molecule (C₅₀ value) which is at the most 50 μM as determined by the assembly binding assay as described herein,

(ii) capable of eliciting INF-γ-producing cells in a Peripheral Blood Leukocytes (“PBL”) population of a cancer patient at a frequency of at least 1 per 10⁴ PBLs as determined by an ELISPOT assay, and/or

(iii) capable of in situ detection in a tumour tissue of CTLs that are reactive with the epitope peptide.

Preferably, the peptide of the invention has at least two, most preferably all of these three features.

In further aspects the invention provides a pharmaceutical composition and a composition for ex vivo or in situ diagnosis of the presence in a cancer patient of survivin reactive T-cells among PBLs or in tumour tissue, which composition comprises a peptide as defined above.

In yet further aspects the invention relates to a diagnostic kit for ex vivo or in situ diagnosis of the presence in a cancer patient of survivin reactive T-cells among PBLs or in tumor tissue, which kit comprises a peptide according of the invention, and a complex of such a peptide and a Class I HLA molecule or a fragment of such molecule.

In another aspect there is also provided a method of detecting in a cancer patient the presence of survivin reactive T-cells, the method comprising contacting a tumour tissue or a blood sample with a complex as defined above and detecting binding of the complex to the tissue or the blood cells.

In still further aspects the invention pertains to a molecule that is capable of binding specifically to a peptide of the invention such as an antibody or a fragment hereof, and to a molecule that is capable of blocking the binding of such a molecule.

The invention also provides a method of treating a cancer disease, the method comprising administering to a patient suffering from the disease an effective amount of the pharmaceutical composition of the invention, a molecule of the invention that is capable of binding specifically to a peptide of the invention or a molecule that is capable of blocking the binding of such a molecule.

### DETAILED DISCLOSURE OF THE INVENTION

The novel MHC Class I-restricted peptide of the invention is characterised by having at least one of several features, one of which is the ability to bind to the Class I HLA molecule to which it is restricted at an affinity, which, when it is measured by the amount of the peptide that is capable of half maximal recovery of the Class I HLA molecule (C₅₀ value) in an assembly assay as described herein, is at the most 50 μM. This assembly assay is carried out as described previously (12,13), and it is based on stabilisation of the HLA molecule after loading of peptide to the peptide transporter deficient cell line T2. Subsequently, correctly folded stable HLA heavy chains are immunoprecipitated using conformation dependent antibodies and the peptide binding is quantitated.

This assay provides a simple means of screening candidate peptides for their ability to bind to a given HLA allele molecule at the above affinity. In preferred embodiments, the peptide of the invention in one having a C₅₀ value, which is at the most 30 μM, such as a C₅₀ value, which is at the most 20 μM including C₅₀ values of at the most 10 μM, at the most 5 μM and at the most 2 μM.

As mentioned above, the HLA system represents the human major histocompatibility (MHC) system. Generally, MHC systems control a range of characteristics: transplantation antigens, thymus dependent immune responses, certain complement factors and predisposition for certain diseases. More specifically, the MHC codes for three different types of molecules, i.e. Class I, II and III molecules, which determine the more general characteristics of the MHC. Of these molecules, the Class I molecules are so-called HLA-A, HLA-B and HLA-C molecules that are presented on the surface of most nucleated cells and thrombocytes.

The peptides of the present invention are characterised by their ability to bind to (being restricted by) a particular MHC Class I HLA molecule. Thus, in one embodiment the peptide is one which is restricted by a MHC Class I HLA-A molecule including HLA-A1, HLA-A2, HLA-A3, HLA-A9, HLA-A10, HLA-A11, HLA-Aw19, HLA-A23(9), HLA-A24(9), HLA-A25(10), HLA-A26(10),, HLA-A28, HLA-A29(w19), HLA-A30(w19), HLA-A31(w19), HLA-A32(w19), HLA-Aw33(w19), HLA-Aw34(10), HLA-Aw36, HLA-Aw43, HLA-Aw66(10), HLA-Aw68(28), HLA-A69(28). More simple designations are also used throughout the literature, where only the primary numeric designation is used, e.g. HLA-A19 or HLA-A24 instead of HLA-Aw19 and HLA-A24(9), respectively. In specific embodiments, the peptide of the invention is restricted a MHC Class I HLA species selected from the group consisting of HLA-A1, HLA-A2, HLA-A3, HLA-A11 and HLA-A24.

The peptides of the invention are derived from the known sequence of survivin, e.g. the sequence disclosed in US 6,245,523. The selection of peptides potentially having the ability to bind to a particular HLA molecule can be made by the alignment of known sequences that bind to a given particular HLA molecule to thereby reveal the predominance of a few related amino acids at particular positions in the peptides. Such predominant amino acid residues are also referred to herein as “anchor residues” or “anchor residue motifs”. By following such a relatively simple procedure based on known sequence data that can be found in accessible databases, peptides can be derived from the survivin protein molecule which are likely to bind to the particular HLA molecule. Representative examples of such analyses for a range of HLA molecules are given in the below table:

| HLA | | | | | | | C- |
|---|---|---|---|---|---|---|---|
| allele | Position 1 | Position 2 | Position 3 | Position 5 | Position 6 | Position 7 | terminal |
| HLA-A1 | | T, S | D, E | | | L | Y |
| HLA-A2 | | L, M | | | V | | L, V |
| HLA-A3 | | L, V, M | F, Y | | | | K, Y, F |
| HLA-A11 | | V, I, F, Y | M, L, F, Y, I | | | | K, R |
| HLA-A23 | | I, Y | | | | | W, I |
| HLA-A24 | | Y | | I, V | F | | I, L, F |
| HLA-A25 | | M, A, T | I | | | | W |
| HLA-A26 | E, D | V, T, I, L, F | | | I, L, V | | Y, F |
| HLA-A28 | E, D | V, A, L | | | | | A, R |
| HLA-A29 | | E | | | | | Y, L |
| HLA-A30 | | Y, L, F, V | | | | | Y |
| HLA-A31 | | | L, M, F, Y | | | | R |
| HLA-A32 | | I, L | | | | | W |
| HLA-A33 | | Y, I, L, V | | | | | R |
| HLA-A34 | | V, L | | | | | R |
| HLA-A66 | E, D | T, V | | | | | R, K |
| HLA-A68 | E, D | T, V | | | | | R, K |
| HLA-A69 | | V, T, A | | | | | V, L |
| HLA-A74 | | T | | | | | V, L |
| HLA-B5 | | A, P | F, Y | | | | I, L |
| HLA-B7 | | P | | | | | L, F |
| HLA-B8 | | | K | K, R | | | L |
| HLA-B14 | | R, K | | | | | L, V |
| HLA-B15 | | Q, L, K, P, H, | | | | | F, Y, W |
| (B62) | | V, I, M, S, T | | | | | |
| HLA-B17 | | | | | | | L, V |
| HLA-B27 | | R | | | | | Y, K, F, L |
| HLA-B35 | | P | | | | | I, L, M, Y |
| HLA-B37 | | D, E | | | | | I, L, M |
| HLA-B38 | | H | D, E | | | | F, L |
| HLA-B39 | | R, H | | | | | L, F |
| HLA-B40 | | E | F, I, V | | | | L, V, A, W, M, |
| (B60, 61) | | | | | | | T, R |
| HLA-B42 | | L, P | | | | | Y, L |
| HLA-B44 | | E | | | | | F, Y, W |
| HLA-B46 | | M, I, L, V | | | | | Y, F |
| HLA-B48 | | Q, K | | | | | L |
| HLA-B51 | | A, P, G | | | | | F, Y, I, V |
| HLA-B52 | | Q | F, Y | | | | I, V |
| HLA-B53 | | P | | | | | W, F, L |
| HLA-B54 | | P | | | | | |
| HLA-B55 | | P | | | | | A, V |
| HLA-B56 | | P | | | | | A, V |
| HLA-B57 | | A, T, S | | | | | F, W, Y |
| HLA-B58 | | A, T, S | | | | | F, W, Y |
| HLA-B67 | | P | | | | | L |
| HLA-B73 | | R | | | | | P |
| HLA-Cw1 | | A, L | | | | | L |
| HLA-Cw2 | | A, L | | | | | F, Y |
| HLA-Cw3 | | A, L | | | | | L, M |
| HLA-Cw4 | | Y, P, F | | | | | L, M, F, Y |
| HLA-Cw6 | | | | | | | L, I, V, Y |
| HLA-Cw6 | | Y | | | | | L, Y, F |
| HLA-Cw8 | | Y | | | | | L, I, |
| HLA-Cw16 | | A, L | | | | | L, V |

Thus, as an example, nonapeptides potentially having the ability to bind to HLA-A1 would have one of the following sequences: Xaa-T-D-Xaa-Xaa-Xaa-L-Xaa-Y, Xaa-T-E-Xaa-Xaa-Xaa-L-Xaa-Y; Xaa-S-D-Xaa-Xaa-Xaa-L-Xaa-Y or Xaa-S-E-Xaa-Xaa-Xaa-L-Xaa-Y (Xaa indicating any amino acid residue). In a similar manner, sequences potentially having the ability to bind to any other HLA molecule can be designed.

It will be appreciated that the person of ordinary skill in the art will be able to identify further “anchor residue motifs” for a given HLA molecule.

Thus, in useful embodiments, the peptides of the invention include peptides, the sequences of which comprise, for each of the specific HLA alleles listed in the table, any of the amino acid residues as indicated in the table.

Thus, a simple approach to identifying peptides of the invention includes the following steps: selecting a particular HLA molecule, e.g. one occurring at a high rate in a given population, carrying out an alignment analysis as described above to identify “anchor residue motifs” in the survivin protein, isolating or constructing peptides of a suitable size that comprise one or more of the identified anchor residues and testing the resulting peptides for (i) capability to bind to the particular HLA molecule using the assembly assay as described herein, (ii) the capability of the peptides to elicit INF-γ-producing cells in a PBL population of a cancer patient at a frequency of at least 1 per 10⁴ PBLs as determined by an ELISPOT assay as described herein, and/or (iii) the capability of the peptides to detect in situ in a tumour tissue CTLs that are reactive with the epitope peptides being tested.

In specific embodiments, the peptide of the invention is an HLA-A2 restricted survivin-derived peptide having a sequence selected from the following: FLKLDRERA (survivin₁₀₁₋₁₀₉) (SEQ ID NO:1), TLPPAWQPFL (survivin₅₋₁₄) (SEQ ID NO:2), ELTLGEFLKL (survivin₉₅₋₁₀₄) (SEQ ID NO:3), LLLGEFLKL (SEQ ID NO:4) and LMLGEFLKL (SEQ ID NO:5). (The designations in brackets indicate the positions of the residues in the survivin protein as disclosed in U.S. Pat. No. 6,245,523). LLLGEFLKL (SEQ ID NO:4) is a sequence derived from survivin₉₆₋₁₀₄ by substituting “T” in position 2 of the peptide with an “L” and LMLGEFLKL (SEQ ID NO:5) is derived from survivin₉₆₋₁₀₄ by substituting “T” in position 2 with “M”.

In further useful embodiments, the peptide of the invention is a peptide, which is restricted by a MHC Class I HLA-B molecule including any of the following: HLA-B5, HLA-B7, HLA-B8, HLA-B12, HLA-B13, HLA-B14, HLA-B15, HLA-B16, HLA-B17, HLA-B18, HLA-B21, HLA-Bw22, HLA-B27, HLA-B35, HLA-B37, HLA-B38, HLA-B39, HLA-B40, HLA-Bw41, HLA-Bw42, HLA-B44, HLA-B45, HLA-Bw46 and HLA-Bw47. In specific embodiments, the MHC Class I HLA-B species to which the peptide of the invention is capable of binding is selected from HLA-B7, HLA-B35, HLA-B44, HLA-B8, HLA-B15, HLA-B27 and HLA-B51.

In specific embodiments, the peptide of the invention is an HLA-B35-restricted survivin-derived peptide having a sequence selected from the following: CPTENEPDL (survivin₄₆₋₅₄) (SEQ ID NO:6), EPDLAQCFF (survivin₅₁₋₅₉) (SEQ ID NO:7), CPTENEPDY (SEQ ID NO:8) and EPDLAQCFY (SEQ ID NO:9). (The designations in brackets indicate the positions of the residues in the survivin protein as disclosed in U.S. Pat. No. 6,245,523). CPTENEPDY (SEQ ID NO:8) is a sequence derived from survivin₄₆₋₅₄ by substituting “L” in the C-terminal of the peptide with a “Y” and EPDLAQCFY (SEQ ID NO:9) is derived from survivin₅₁₋₅₉ by substituting an “F” residue in the C-terminal 2 with a “Y”.

In further useful embodiments, the peptide of the invention is a peptide, which is restricted by a MHC Class I HLA-C molecule including any of the following: HLA-Cw1, HLA-Cw2, HLA-Cw3, HLA-Cw4, HLA-Cw5, HLA-Cw6, HLA-Cw7 and HLA-Cw1.

Preferably, the peptide of the invention comprises less than 50 amino acid residues, and more preferably it comprises at the most 20 amino acid residues, such as at the most 10 amino acid residues. In specific embodiments, the peptide is a heptapeptide, an octopeptide, a nonapeptide, a decapeptide or an undecapeptide.

The peptide of the invention is, as mentioned above, derived from a survivin protein or a fragment hereof. The survivin protein from which the peptide can be derived is survivin protein from any animal species in which the protein is expressed. In preferred embodiments, the survivin starting protein is from a mammal species including a rodent species, rabbit and a primate species such as humans. Based on the sequence of the selected survivin protein, the peptide of the invention is derived by any appropriate chemical or enzymatic treatment of the survivin starting material that results in a peptide of a suitable size as indicated above, or it can be synthesised by any conventional peptide synthesis procedures with which the person of ordinary skills in the art is familiar.

The peptide of the invention may have a sequence which is a native sequence of the survivin protein from which is derived. However, peptides having a higher affinity to any given HLA molecule may be derived from such a native sequence by modifying the sequence by substituting, deleting or adding at least one amino acid residue, e.g. on the basis of the procedure described above whereby anchor residue motifs in respect of the given HLA molecule are identified.

A significant feature of the peptide of the invention is its capability to recognise or elicit INF-γ-producing responder T cells, i.e. cytotoxic T cells (CTLs) that specifically recognise the particular peptide in a PBL population or tumour cells of a cancer patient (target cells). This activity is readily determined by subjecting PBLs or tumour cells from a patient to an ELISPOT assay as described in reference (16) and in the following examples. Prior to the assay, it may be advantageous to stimulate the PBL population or the tumour cells to be assayed by contacting the cells with the peptide to be tested. Preferably, the peptide is capable of eliciting or recognising INF-Y -producing T cells at a frequency of at least 1 per 10⁴ PBLs as determined by an ELISPOT assay as used herein. More preferably the frequency is at least 5 per 10⁴ PBLs, most preferably at least 10 per 10⁴ PBLs, such as at least 50 or 100 per 10⁴ PBLs.

The ELISPOT assay represents a strong tool to monitor survivin peptide specific T-cell responses. However, although it has been shown that ELISPOT reactivity in most cases correlates with the capacity of the CLLs to lyse target cells, the conclusive evidence for this notion can only be given directly. Such direct evidence is provided herein, as it was demonstrated (see Example 2) that survivin reactive cells isolated by means of HLA/peptide complexes possess the functional capacity of lysing target cells. Additionally, it was demonstrated that the isolated CTLs specifically recognising a peptide of the invention were capable of lysing HLA-matched tumour cells of different origin, e.g. melanomas and breast cancer. This finding strongly suggests that cancer cells in general process and present the same endogenous survivin peptide. Therefore, a major implication of the findings herein is that the peptides of the invention are expressed and complexed with HLA molecules on a variety of cancer cells of different histological origins. This renders these cancer cells susceptible to destruction by CTLs and emphasizes the potential usefulness of survivin immunization to control the growth of different neoplasms. The presence of spontaneous CTL-responses in PBLs and tumour cells to HLA-restricted survivin-derived peptide epitopes from patients suffering from three unrelated cancer types, i.e., breast cancer, melanoma and CLL, further substantiates the universal immunotherapeutic potential of this tumour antigen.

Accordingly, in another preferred embodiment the peptide of the invention is capable of eliciting INF-γ-producing cells in a PBL population of a patient having a cancer disease where survivin is expressed including a haematopoietic malignancy including chronic lymphatic leukemia and chronic myeloid leukemia, melanoma, breast cancer, cervix cancer, ovary cancer, lung cancer, colon cancer, pancreas cancer and prostate cancer. Specifically, the peptide of the invention is able to elicit an immune response in the form of T cell having cytotoxic effect against survivin expressing cells of a cancer cell line, including a cell line selected from the group consisting of the breast cancer cell line MCF-7 and the melanoma cell line FM3.

In addition to their capacity to elicit immune responses in PBL populations and cancer cell lines, it was demonstrated that the peptides of the invention are also capable of eliciting cytolytic immune responses in situ, i.e. in solid tumour tissues. This was demonstrated by providing HLA-peptide complexes, e.g. being multimerised and being provided with a detectable label, and using such complexes for immunohistochemistry stainings to detect in a tumour tissue CTLs that are reactive with the epitope peptide of the invention. Accordingly, a further significant feature of the peptide of the invention is that it is capable of in situ detection in a tumour tissue of CTLs that are reactive with the epitope peptide.

It is contemplated that the peptides of the invention, in addition to their capacity to bind to HLA molecules resulting in the presentation of complexes of HLA and peptides on cell surfaces, which complexes in turn act as epitopes or targets for cytolytic T cells, may elicit other types of immune responses, such as B-cell responses resulting in the production of antibodies against the complexes and/or a Delayed Type Hypersensitivity (DTH) reaction. The latter type of immune response is defined as a redness and palpable induration at the site of injection of the peptide of the invention.

It is evident that the findings of the present invention provide the basis for therapeutic as well as diagnostic applications of the survivin derived peptides.

Accordingly, in a further aspect the present invention provides a pharmaceutical composition comprising the peptide of the invention. As the peptides of the invention are relatively small molecules it may be required in such compositions to combine the peptides with various materials such as adjuvants, to produce vaccines, immunogenic compositions, etc. Adjuvants, broadly defined, are substances which promote immune responses. Frequently, the adjuvant of choice is Freund's complete or incomplete adjuvant, or killed B. pertussis organisms, used e.g. in combination with alum precipitated antigen. A general discussion of adjuvants is provided in Goding, Monoclonal Antibodies: Principles & Practice (2nd edition, 1986) at pages 61-63. Goding notes, however, that when the antigen of interest is of low molecular weight, or is poorly immunogenic, coupling to an immunogenic carrier is recommended. Examples of such carrier molecules include keyhole limpet haemocyanin, bovine serum albumin, ovalbumin and fowl immunoglobulin. Various saponin extracts have also been suggested to be useful as adjuvants in immunogenic compositions. Recently, it has been proposed to use granulocyte-macrophage colony stimulating factor (GM-CSF), a well known cytokine, as an adjuvant (WO 97/28816). Accordingly, the invention encompasses a therapeutic composition further comprising any adjuvant substance including any of the above or combinations thereof. It is also contemplated that the antigen, i.e. the peptide of the invention and the adjuvant can be administered separately in any appropriate sequence.

The choice of antigen in the pharmaceutical composition of the invention will depend on parameters determinable by the person of skill in the art. As it has been mentioned, each of the different peptides of the invention is presented on the cell surfaces by a particular HLA molecule. As such, if a subject to be treated is typed with respect to HLA phenotype, a peptide/peptides are selected that is/are known to bind to that particular HLA molecule.

Alternatively, the antigen of interest is selected based on the prevalence of the various HLA phenotypes in a given population. As an example, HLA-A2 is the most prevalent phenotype in the Caucasian population, and therefore, a composition containing a survivin derived peptide binding to HLA-A2 will be active in a large proportion of that population. However, the composition of the invention may also contain a combination of two or more survivin derived peptides, each interacting specifically with a different HLA molecule so as to cover a larger proportion of the target population. Thus, as examples, the pharmaceutical composition may contain a combination of a peptide restricted by a HLA-A molecule and a peptide restricted by a HLA-B molecule, e.g. including those HLA-A and HLA-B molecules that correspond to the prevalence of HLA phenotypes in the target population, such as e.g. HLA-A2 and HLA-B35. Additionally, the composition may comprise a peptide restricted by an HLA-C molecule.

It is complated that useful immunogenic compositions of the inventions in addition to a survivin derived peptide as defined herein may comprise an immunologically effective amount of the survivin protein as such as it is defined herein or an immunogenic fragment hereof.

The amount of the immunogenic peptide of the invention in the pharmaceutical composition may vary, depending on the particular application. However, a single dose of the immunogen is preferably anywhere from about 10 μg to about 5000 μg, more preferably from about 50 μg to about 2500 μg such as about 100 μg to about 1000 μg. Modes of administration include intradermal, subcutaneous and intravenous administration, implantation in the form of a time release formulation, etc. Any and all forms of administration known to the art are encompassed herein. Also any and all conventional dosage forms that are known in the art to be appropriate for formulating injectable immunogenic peptide composition are encompassed, such as lyophilised forms and solutions, suspensions or emulsion forms containing, if required, conventional pharmaceutically acceptable carriers, diluents, preservatives, adjuvants, buffer components, etc.

The immunoprotective effect of the composition of the invention can be determined using several approaches. Examples hereof are provided in the following examples. A further example on how to determine a CTL response provoked by the immunogenic composition is provided in WO 97/28816, supra. A successful immune response may also be determined by the occurrence of DTH reactions after immunisation and/or the detection of antibodies specifically recognising the peptide(s) of the vaccine composition.

In preferred embodiments, the pharmaceutical composition of the invention is an immunogenic composition or vaccine capable of eliciting an immune response to a cancer disease. As used herein, the expression “immunogenic composition or vaccine” refers to a composition eliciting at least one type of immune response directed against cancer cells. Thus, such an immune response may be any of the types mentioned above: A CTL response where CTLs are generated that are capable of recognising the HLA/peptide complex presented on cell surfaces resulting in cell lysis, i.e. the vaccine elicits the production in the vaccinated subject of effector T-cells having a cytotoxic effect against the cancer cells; a B-cell response giving rise to the production of anti-cancer antibodies; and/or a DTH type of immune response.

In useful embodiments an immunogenic response directed against a cancer disease is elicited by administering the peptide of the invention either by loading MHC class I molecules on antigen presenting cells (APCs) from the patient, by isolating PBLs from the patient and incubating the cells with the peptide prior to injecting the cells back into the patient or by isolating precursor APCs from the patient and differentiating the cells into professional APCs using cytokines and antigen before injecting the cells back into the patient. Thus, in one embodiment of the present invention, a method for treating cancer patients is one wherein the peptide is administered by presenting the peptide to the patient's antigen presenting cells (APCs) ex vivo followed by injecting the thus treated APCs back into the patient. There are at least two alternative ways of performing this. One alternative is to isolate APCs from the cancer patient and incubate (load) the MHC class I molecules with the peptide. Loading the MHC class I molecules means incubating the APCs with the peptide so that the APCs with MHC class I molecules specific for the peptide will bind the peptide and therfore be able to present it to T cells. Subsequently, the APCs are re-injected into the patient. Another alternative way relies on the recent discoveries made in the field of dendritic cell biology. In this case, monocytes (being dendritic cell precursors) are isolated from the patient and differentiated in vitro into professional APC (or dendritic cells) by use of cytokines and antigen. This is described in Examples 3 and 5, where adherent PBLs (being mainly monocytes) are cultured in vitro together with GM-CSF, IL-4 and TNF-α. Subsequently, the in vitro generated DCs are pulsed with the peptide and injected into the patient.

Due to the fact that survivin appears to be expressed in most cancer forms, it is very likely that vaccines of the invention can be provided to control any type of cancer disease where survivin is expressed. Thus, as examples, the vaccine composition of the invention is immunologically active against a haematopoietic malignancy including chronic lymphatic leukemia and chronic myeloid leukemia, melanoma, breast cancer, cervix cancer, ovary cancer, lung cancer, colon cancer, pancreas cancer and prostate cancer.

From the above description, the skilled person will readily realise that the peptides of the invention are useful as cancer diagnostic tools, particularly so, as the peptides are derived from survivin expressed in all cancer types. Therefore, the peptides of the invention provide the basis for developing universally applicable diagnostic and prognostic procedures in respect of cancer diseases. Thus, in other useful embodiments the composition of the invention is a composition for ex vivo or in situ diagnosis of the presence in a cancer patient, e.g. based on the detection of survivin reactive T-cells among PBLs or in tumour tissue.

Accordingly, there is, in still further aspects, provided a diagnostic kit for ex vivo or in situ diagnosis of the presence in a cancer patient of survivin reactive T-cells among PBLs or in tumor tissue comprising one or more peptides of the invention, and a method of detecting in a cancer patient the presence of survivin reactive T-cells, the method comprising contacting a tumor tissue or a blood sample with a complex of a peptide of the invention and a Class I HLA molecule or a fragment of such molecule and detecting binding of the complex to the tissue or the blood cells.

Another useful diagnostic or prognostic approach is based on generating antibodies in a heterologous animal species, e.g. murine antibodies directed against a human survivin derived peptide of the invention, which can then be used, e.g. to diagnose for the presence of cancer cells presenting the peptide. For such immunisation purposes, the amount of peptide may be less than that used in the course of in vivo therapy, such as that mentioned above. In general, a preferred dose can range from about 1 μg to about 750 μg of peptide. It is also possible to produce monoclonal antibodies based on immunisation with a peptide of the invention. Accordingly, the present invention also relates to a molecule, in particular a monoclonal or polyclonal antibody including a fragment hereof, that is capable of binding specifically to a peptide of the invention and to a molecule that is capable of blocking such a binding, e.g. an antibody raised against the monoclonal or polyclonal antibody directed against a peptide of the invention.

In one aspect, the invention provides a complex of a peptide of the invention and a Class I HLA molecule or a fragment of such molecule, which is useful as a diagnostic reagent such as it is described supra. The complex is made by any conventional means including those described in the following examples. Such a complex may be monomeric or multimeric.

The present invention provides the means for alleviating or curing a cancer disease. Accordingly, it is a further aspect of the invention to provide a method of treating a cancer disease associated with the expression of survivin, including as examples: a haematopoietic malignancy including chronic lymphatic leukemia and chronic myeloid leukemia, melanoma, breast cancer, cervix cancer, ovary cancer, lung cancer, colon cancer, pancreas cancer and prostate cancer, which method comprises administering to a patient suffering from the disease an effective amount of the pharmaceutical composition according to the invention, a molecule that is capable of binding specifically to a peptide of the invention and/or a molecule that is capable of blocking the binding of such a molecule.

In some cases it will be appropriate to combine the treatment method of the invention with a conventional cancer treatment such as radiotherapy or chemotherapy.

The invention will now be described in further details in the below, non-limiting examples and the figures, wherein

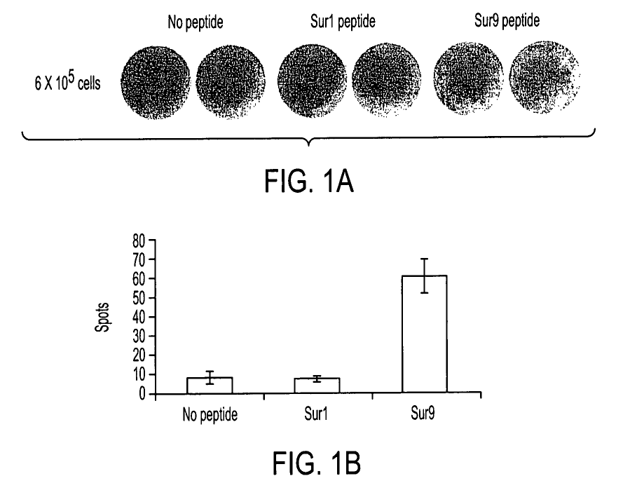

FIG. 1 illustrates T-cell response as measured in an ELISPOT in patient CLL1 to no peptide, Sur1 (LTLGEFLKL, SEQ ID NO:10) peptide and Sur9 (ELTLGEFLKL, SEQ ID NO:3) peptide. PBLs were stimulated once with peptide before plated at 6×10⁵ cells per well in duplicate (A). The average number of spots per peptide was calculated using a CCD scanning device and a computer system (B),

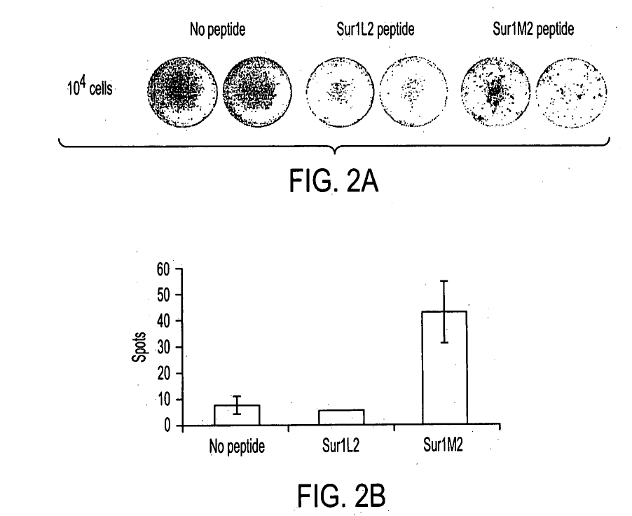

FIG. 2 illustrates T-cell response as measured in an ELISPOT in patient CLL1 to no peptide, the peptide analogue Sur1L2 (LLLGEFLKL, SEQ ID NO:4), and the peptide analogue Sur1M2 (LMLGEFLKL, SEQ ID NO:5). PBLs were stimulated once with peptide before plated at 10⁴ cells per well in duplicate (A). The average number of spots per peptide was calculated using a CCD scanning device and a computer system (B),

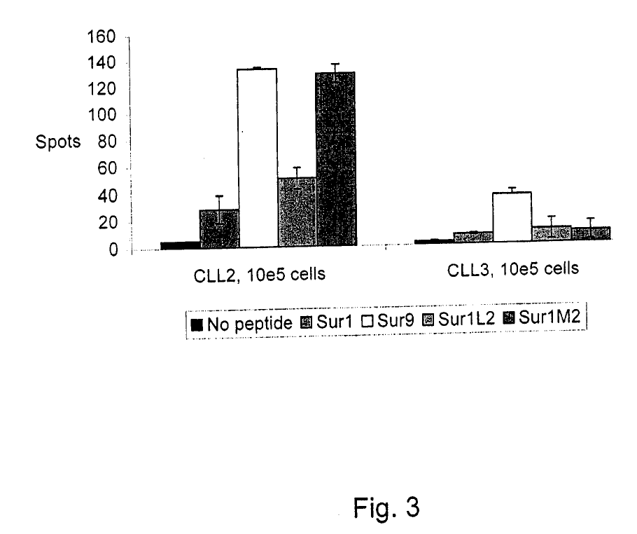

FIG. 3 shows responses as measured in an ELISPOT in patient CLL2 and CLL3 to no peptide (black bar), the Sur1 (LTLGEFLKL, SEQ ID NO:10) peptide (grey bar), the Sur9 (ELTLGEFLKL, SEQ ID NO:3) peptide (white bar), the analogue peptide Sur1L2 (LLLGEFLKL, SEQ ID NO:4) (light grey bar), and the analogue peptide Sur1M2 (LMLGEFLKL, SEQ ID NO:5) (dark grey bar). Each experiment was performed with 10⁵ cells per well in duplicate, and the average number of spots was calculated,

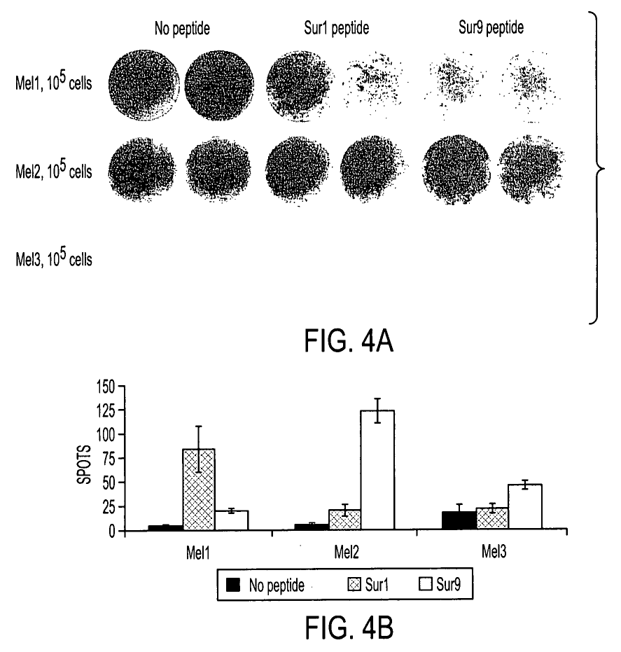

FIG. 4 represents T cells that were isolated from tumour infiltrated lymph nodes from patient Mel1 (A, top row), Mel2 (A, middle row), and Mel3 (A, bottom row), stimulated once in vitro and analyzed in an ELISPOT assay for response to the peptides Sur1 (LTLGEFLKL, SEQ ID NO: 10) and Sur9 (ELTLGEFLKL, SEQ ID NO:3). Each experiment was performed in duplicate with 10⁵ cells per well. In each experiment two wells without addition of peptide was also included (A). The average number of spots per peptide was calculated for each patient (B),

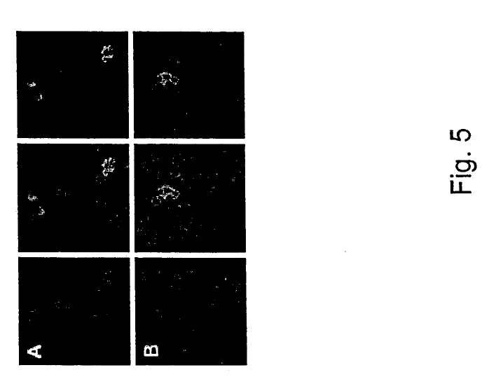

FIG. 5 illustrates in situ detection of survivin-reactive CTLs. (A) Confocal laser scanning microscopy was used to detect CTLs reacting with an Cy3-conjugated anti-CD8 antibody (red channel) and/or an FITC-conjugated multimeric MHC/survivin-peptide construct (green channel) in a primary tumor from a stage III melanoma patient. (B) Staining with an anti-CD8 antibody (red channel) and a FITC-conjugated multimeric MHC/survivin-peptide construct (green channel) in a sentinel lymph node from the same patient,

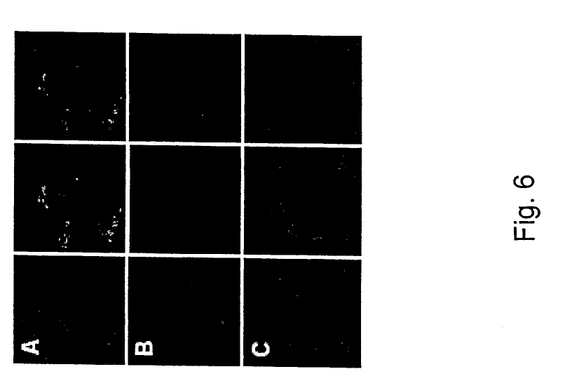

FIG. 6 shows in situ detection of survivin-reactive CTLs. (A) Confocal laser scanning microscopy was used to detect CTLs reacting with an Cy3-conjugated anti-CD8 antibody (red channel) and a FITC-conjugated multimeric MHC/survivin-peptide construct (green channel) in a breast cancer metastasis from a HLA-A2 positive patient. (B) Staining with an anti-CD8 antibody (red channel) and an FITC-conjugated multimeric MHC/gp100-peptide construct (green channel) in a breast cancer metastasis from an HLA-A2 positive patient. (C) Staining with an anti-CD8 antibody (red channel) and a FITC-conjugated multimeric MHC/survivin-peptide construct (green channel) in a breast cancer metastasis from a HLA-A2 negative patient,

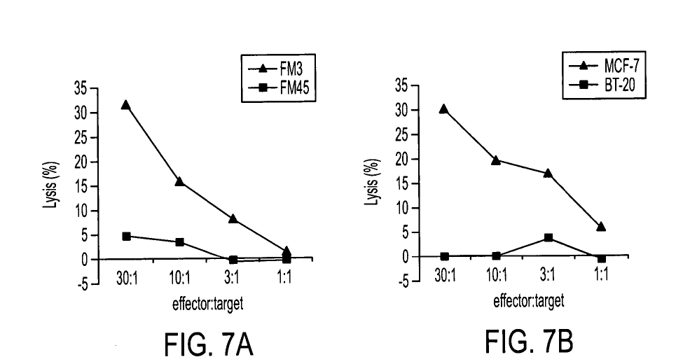

FIG. 7 shows functional activity of survivin specific CTLs. CTLs were isolated from a melanoma infiltrated lymph node using survivin coated magnetic beads. (A) Specific lysis of melanoma cell lines; the HLA-A2 positive FM3 (triangle) and the HLA-A2 negative FM45 (square). (B) Specific lysis of breast cancer cell lines; the HLA-A2 positive MCF-7 (triangle) and the HLA-A2 negative BT-20 (square),

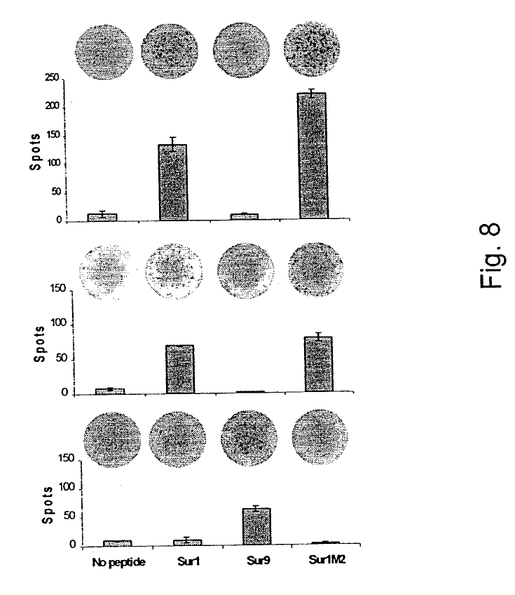

FIG. 8 shows frequency of survivin reactive CTLs in PBL from breast cancer patients. Reactivity was examined in three breast cancer patients (top, middle, and bottom, respectively) by the ELISPOT. For each patient the first well represents assays performed in the absence of peptide, the second well in the presence of surl peptide, the third well in the presence of sur9, and the fourth well in the presence of the modified sur1M2 peptide. 1×10⁴ effector cells per well were used. The graph depicts the quantification of reactive cells; grey columns represent the average number of IFN-γ producing cells,

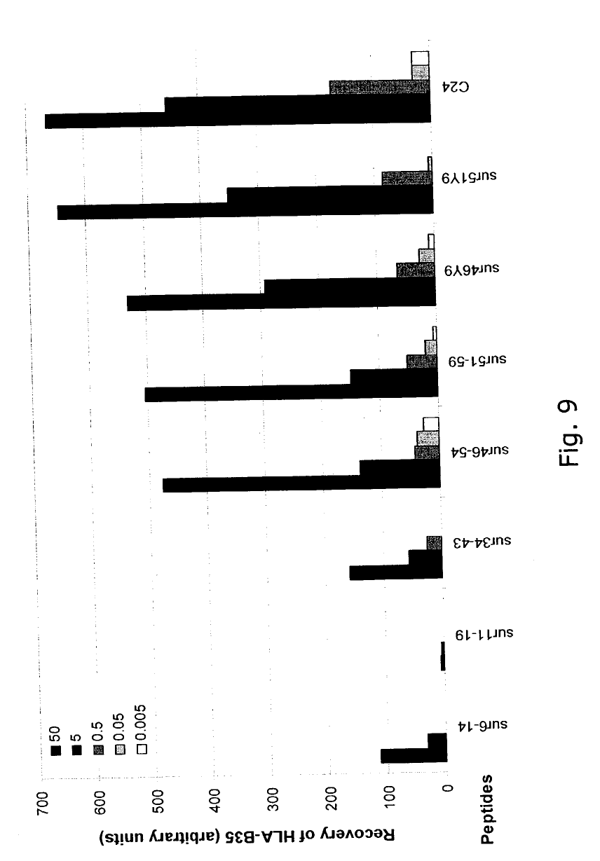

FIG. 9 illustrates HLA-35 binding of survivin derived peptides and analysis of the peptide-mediated recovery of HLA-B35 molecules by survivin derived peptides. Lysates of metabolically labeled T2-B35 cells were incubated at 4° C. in the presence of 50, 5, 0.5, 0.05 and 0.005 mM of peptide. The recovery of HLA-B35 was analyzed in an assembly assay and quantified subsequent to IEF-gel electrophoresis, using ImageGauge phosphorimager software (FUJI photo film Co., LTD., Japan). The C₅₀ value is the concentration of the peptide required for half-maximal binding to HLA-B35,

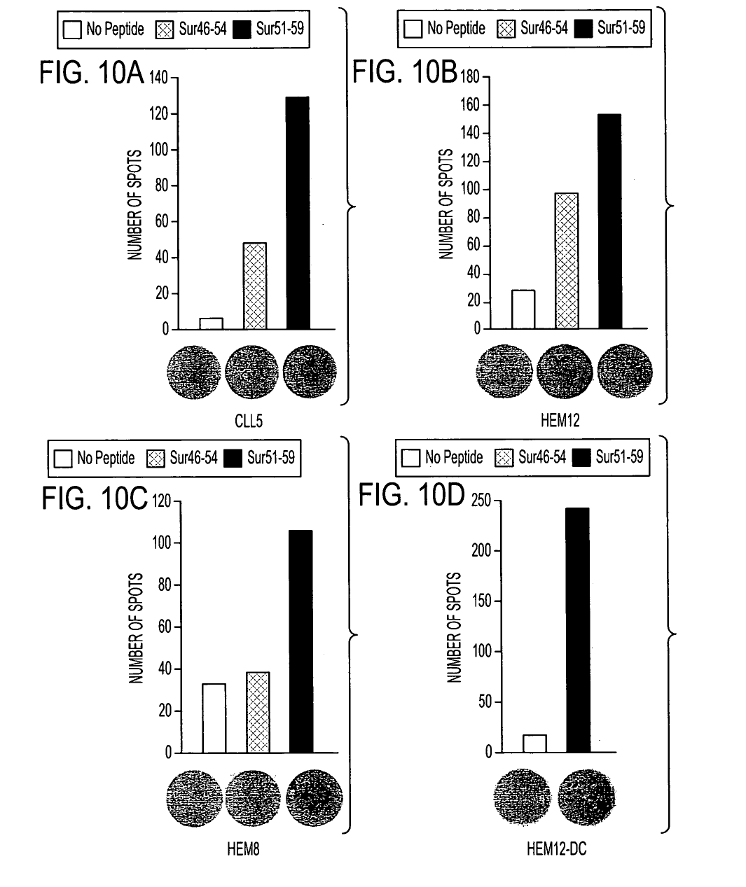

FIG. 10 shows spontaneous T-cell responses observed in PBLs from cancer patients. A) The number of IFNγ spot forming cells measured in ELISPOT assay without peptide, with sur51-59 or sur46-54, among in vitro stimulated PBLs from patient CLL5 (10⁵ cells/well), HEM12 (10⁵ cells/well), and HEM8 (5×10⁴ cells/well). B) The number of spot forming cells among 1.7×10⁵ PBLs from HEM12, cultured for 10 days with peptide-pulsed matured autologous dendritic cells. The columns represent the average of two measurements,

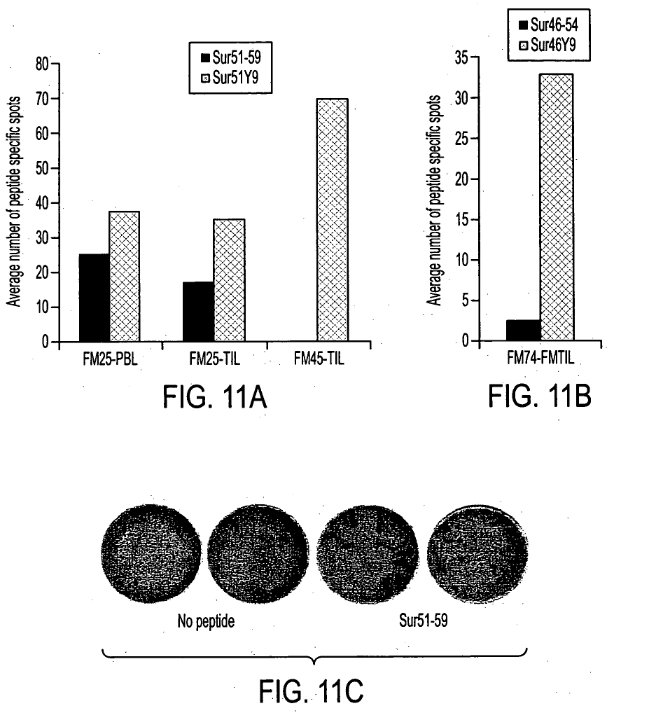

FIG. 11 demonstrates spontaneous T-cell responses against native and modified survivin peptides in melanoma patients. A) The number of spot forming cells measured in ELISPOT assay against sur51-59 and sur51Y9 from patient FM25 in PBLs (4×10³ cells/well) and TILs (7×10⁴ cells/well) as well as TILs from FM45 (10⁴ cells/well). B) The number of spot forming cells measured in ELISPOT assay against sur46 and sur46Y9 measured in TILs from FM74 (5×10³ cells/well). The columns represent the average of two measurements with the non-specific IFNγ release subtracted. C) ELISPOT assay measuring the IFNγ release of sur51Y9/HLA-B35 isolated cells in response to the native peptide sur51-59, and

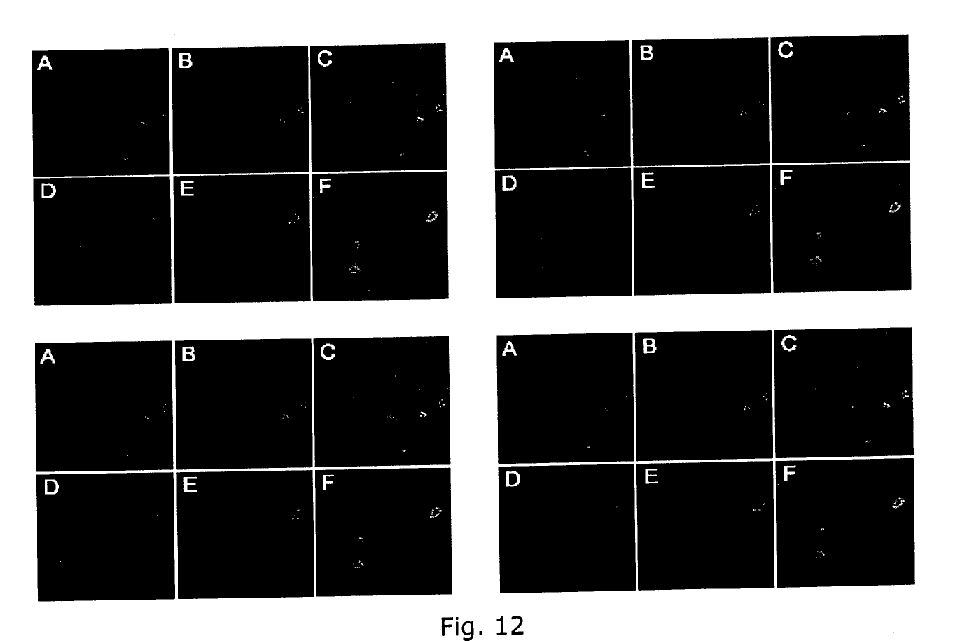

FIG. 12 shows in situ staining of a primary melanoma lesion with multimerised sur51Y9/HLA-B35 complexes. Confocal laser scanning microscopy was used to detect CTLs in a primary tumor from a melanoma patient reacting with A) a Cy3-conjugated anti-CD8 40 antibody (red channel), B) an FITC-conjugated multimeric sur51Y9/HLA-B35 construct (green channel), C) an overlay of the two pictures (yellow). D) Staining with a Granzyme B monoclonal antibodies antibody (red channel), E) a FITC-conjugated multimeric MHC/survivin-peptide construct (green channel) and F) an overlay of the two pictures (yellow).

In the following table, amino acid sequences for peptides used herein and their respective SEQ ID NOs are listed:

| | | |
|---|---|---|
| SEQ ID | | |
| NO: | DESIGNATION | SEQUENCE |

| | | |
|---|---|---|
| 1 | Sur6 | FLKLDRERA |
| 2 | Sur8 | TLPPAWQPFL |
| 3 | Sur9 | ELTLGEFLKL |
| 4 | Sur1L2 | LLLGEFLKL |
| 5 | Sur1M2 | LMLGEFLKL |
| 6 | Sur 46-54 | CPTENEPDL |
| 7 | Sur51-59 | EPDLAQCFF |
| 8 | Sur46Y9 | CPTENEPDY |
| 9 | sur51Y9 | EPDLAQCFY |
| 10 | Sur1 | LTLGEFLKL |
| 11 | C1 | ILKEPVHGV |
| 12 | Sur2 | RAIEQLAAM |
| 13 | Sur3 | KVRRAIEQL |
| 14 | Sur4 | STFKNWPFL |
| 15 | Sur5 | SVKKQFEEL |
| 16 | Sur7 | TAKKVRRAI |
| 17 | Sur10 | ETAKKVRRAI |
| 18 | Sur 6-14 | LPPAWQPFL |
| 19 | Sur 11-19 | QPFLKDHRI |
| 20 | Sur 34-43 | TPERMAEAGF |
| 21 | C24 | YPLHEQHQM |
| 22 | Sur14-22 | LKDHRISTF |
| 23 | Sur38-46 | MAEAGFIHC |
| 24 | Sur93-101 | FEELTLGEF |
| 25 | Sur47-56 | PTENEPDLAQ |
| 26 | Sur49-58 | ENEPDLAQCF |
| 27 | Sur92-101 | QFEELTLGEF |
| 28 | C1 | VSDGGPNLY |
| 29 | sur14Y9 | LKDHRISTY |
| 30 | sur93Y9 | FEELTLGEY |
| 31 | sur92Y9 | QFEELTLGEY |
| 32 | sur34Y9 | TPERMAEAGY |
| 33 | sur49Y9 | ENEPDLAQCY |
| 34 | Sur92T2 | QTEELTLGEF |
| 35 | Sur92S2 | QSEELTLGEF |
| 36 | Sur93T2 | FTELTLGEF |
| 37 | Sur93S2 | FSELTLGEF |
| 38 | Sur38Y9 | MAEAGFIHY |
| 39 | Sur46Y10 | PTENEPDLAY |
| 40 | Sur 5-13 | TLPPAWQPF |
| 41 | Sur 53-61 | DLAQCFFCF |
| 42 | Sur 54-62 | LAQCFFCFK |
| 43 | Sur 95-103 | ELTLGEFLK |
| 44 | Sur 112-120 | KIAKETNNK |
| 45 | Sur 13-22 | FLKDHRISTF |
| 46 | Sur 18-26 | RISTFKNWPF |
| 47 | Sur 53-62 | DLAQCFFCFK |
| 48 | Sur 84-92 | CAFLSVKKQF |
| 49 | Sur 101-120 | FLKLDRERAK |
| 50 | Sur 103-112 | KLDRERAKNK |
| 51 | Sur 112-121 | KIAKETNNKK |
| 52 | Sur 113-125 | IAKETNNKKK |
| 53 | C3 | ILRGSVAHK |
| 54 | Sur5K9 | TLPPAWQPK |
| 55 | Sur53K9 | DLAQCFFCK |
| 56 | Sur54L2 | LLQCFFCFK |
| 57 | Sur13K9 | FLKDHRISTK |
| 58 | Sur18K9 | RISTFKNWPK |
| 59 | Sur113L2 | ILKETNNKKK |
| 60 | SurEx3-A3-1 | TIRRKNLRK |
| 61 | SurEx3-A3-2 | PTIRRKNLRK |
| 62 | Sur2b-A3-1 | RITREEHKK |
| 63 | C4 | AVFDRKSDAK |
| 64 | C6 | QPRAPIRPI |
| 65 | C7 | RPPIFIRRL |

### EXAMPLE 1

Identification of a Cytotoxic T-Lymphocyte Response to the Apoptosis Inhibitor Protein Survivin in Cancer Patients

Summary

Using CTL epitopes derived from survivin, specific T-cell reactivity against such antigens in peripheral blood from chronic lymphatic leukemia (CLL) patients and in tumor-infiltrated lymph nodes from melanoma patients by ELISPOT analysis have been studied. CTL responses to survivin derived peptide epitopes were detected in three out of six melanoma patients and in three out of four CLL patients. No T-cell reactivity was detected in PBL from six healthy controls. Thus, survivin derived peptides may serve as important and widely applicable targets for anti-cancer immunotherapeutic strategies.

Introduction

The survivin protein was scanned for the presence of HLA-A*0201 (HLA-A2) binding peptide motifs and after successful identification, the peptides were used to test for specific T-cell reactivity in leukemia and melanoma patients by ELISPOT assay. In both patient cohorts CTL responses to two survivin-derived peptide epitopes were detected, whereas no T-cell reactivity could be detected in the healthy controls. These data suggest that survivin represent a widely expressed tumor antigen recognized by autologous T cells.

Materials and Methods

Patients and Normal Controls

Peripheral vein blood samples from 4 patients diagnosed with CLL (designated CLL1-4) and blood samples from 6 normal individuals were collected into heparinised tubes. PBLs were isolated using Lymphoprep separation and frozen in fetal calf serum (FCS) with 10% dimethylsulphoxide. Additionally, T lymphocytes from tumor-infiltrated lymph nodes were obtained from 6 melanoma patients (designated mel1-6). Freshly resected lymph nodes were minced into small fragments, crushed to release cells into culture and cryopreserved. PBLs were available from 4 of the melanoma patients. All individuals included were HLA-A2 positive as determined by FACS analysis using the HLA-A2 specific antibody BB7.2. The antibody was purified from hybridoma supernatant. Patient samples were obtained from the State University Hospital, Herlev, Denmark. Informed consent was obtained from the patients prior to any of theses measures.

Survivin-Derived Peptides

All peptides were obtained from Research Genetics (Huntsville, Ala., USA) and provided at >90% purity as verified by HPLC and MS analysis. The peptides used are listed in Table 1.

TABLE 1

Peptides examined in this study and their
binding affinity to HLA-A2

| | | | | |
|---|---|---|---|---|
| | | | SEQ ID | |
| Name | Protein^{a} | Sequence | NO: | C₅₀ (μM)^{b} |

| | | | | |
|---|---|---|---|---|
| C1 | HIV-1 pol₄₇₆₋₄₈₄ | ILKEPVHGV | 11 | 0.7 |
| Sur1 | Survivin₉₆₋₁₀₄ | LTLGEFLKL | 10 | >100 |
| Sur2 | Survivin₁₃₃₋₁₄₁ | RAIEQLAAM | 12 | Not |
| | | | | binding |
| Sur3 | Survivin₁₃₀₋₁₃₈ | KVRRAIEQL | 13 | >100 |
| Sur4 | Survivin₂₀₋₂₈ | STFKNWPFL | 14 | Not |
| | | | | binding |
| Sur5 | Survivin₈₈₋₉₆ | SVKKQFEEL | 15 | Not |
| | | | | binding |
| Sur6 | Survivin₁₀₁₋₁₀₉ | FLKLDRERA | 1 | 30 |
| Sur7 | Survivin₁₂₇₋₁₃₅ | TAKKVRRAI | 16 | Not |
| | | | | binding |
| Sur8 | Survivin₅₋₁₄ | TLPPAWQPFL | 2 | 30 |
| Sur9 | Survivin₉₅₋₁₀₄ | ELTLGEFLKL | 3 | 10 |
| Sur10 | Survivin₁₂₆₋₁₃₅ | ETAKKVRRAI | 17 | Not |
| | | | | binding |
| Sur1L2 | | LLLGEFLKL | 4 | 1 |
| Sur1M2 | | LMLGEFLKL | 5 | 1 |

^{a}The value range listed in subscript indicates the position of the peptide in the survivin sequence as disclosed in U.S. Pat No. 6.245.523
^{b}The C₅₀ value is the concentration of the peptide required for half maximal binding to HLA-A2 determined as described below

Assembly Assay for Peptide Binding to Class I MHC Molecules

Assembly assays for binding of the synthetic peptides to class I MHC molecules metabolically labeled with [35S]-methionine were carried out as described (12,13). The assembly assay is based on stabilization of the class I molecules after loading of peptide to the peptide transporter deficient cell line T2. Subsequently, correctly folded stable MHC heavy chains are immunoprecipitated using conformation-dependent antibodies. After IEF electrophoresis, gels were exposed to phosphorimager screens, and peptide binding was quantified using the Imagequant PhosphorImager program (Molecular Dynamics, Sunnyvale, Calif.).

Antigen Stimulation of PBLs

To extend the sensitivity of the ELISPOT assay, PBLs were stimulated once in vitro prior to analysis (14,15). Fresh and previously frozen PBLs gave similar results in the ELISPOT assay. On day 0, PBLs or crushed lymph node were thawed and plated in 2 ml/well at a concentration of 2×10⁶ cells in 24-well plates (Nunc, Denmark) in AIM V medium (Life Technologies, Roskilde, Denmark), 5% heat-inactivated human serum and 2 mM of L-glutamine in the presence of 10 μM of peptide. In each experiment a well without peptide was included. Two days later 300 IU/ml recombinant interleukin-2 (IL-2) (Chiron, Ratingen, Germany) was added to the cultures. The cultured cells were tested for reactivity in the ELISPOT assay on day 12.

ELISPOT Assay

The ELISPOT assay used to quantify peptide epitope-specific interferon-γ-releasing effector cells was performed as in (16). Briefly, nitrocellulose bottomed 96-well plates (MultiScreen MAIP N45, Millipore, Hedehusene, Denmark) were coated with anti-IFN-γ antibody (1-D1K, Mabtech, Nacka, Sweden). The wells were washed, blocked by AIM V medium, and cells were added in duplicates at different cell concentrations. Peptides were then added to each well and the plates were incubated overnight. On the following day, medium was discarded and the wells were washed prior to addition of biotinylated secondary antibody (7-B6-1-Biotin, Mabtech). The plates were incubated for 2 hours, washed and Avidin-enzyme conjugate (AP-Avidin, Calbiochem, Life Technologies) was added to each well. Plates were incubated at RT for 1 hour and the enzyme substrate NBT/BCIP (Gibco, Life Technologies) was added to each well and incubated at room temperature for 5-10 min. The reaction was terminated by washing with tap water upon the emergence of dark purple spots. The spots were counted using the AlphaImager System (Alpha Innotech, San Leandro, Calif. USA) and the peptide specific CTL frequency could be calculated from the numbers of spot-forming cells. The assays were all performed in duplicate for each peptide antigen.

Results

Binding of Survivin Derived Peptides to HLA-A2

The amino acid sequence of the survivin protein was screened for the most probable HLA-A2 nona- and decamer peptide epitopes, using the main HLA-A2 specific anchor residues (17). Ten survivin-derived peptides were synthesized and examined for binding to HLA-A2. An epitope from HIV-1 pol476-484 (ILKEPVHGV, SEQ ID NO:11) (Table 1) was used as a positive control. The peptide concentration required for half maximal recovering of class I MHC (C₅₀ value) was 0.7 μM for the positive control. In comparison, the peptide designated Sur9 (ELTLGEFLKL, SEQ ID NO:3) bound at an affinity of C₅₀=10 μM. The peptides designated Sur6 (FLKLDRERA, SEQ ID NO: 1) and Sur8 (TLPPAWQPFL, SEQ ID NO:2), respectively bound to HLA-A2 at C₅₀=30 μM, whereas Sur1 (LTLGEFLKL, SEQ ID NO:10) and Sur3 (KVRRAIEQL, SEQ ID NO: 13) bound weaker (C₅₀>100 μM). Five of the peptides examined (Sur2, Sur4, Sur5, Sur7, and Sur10) did not bind to HLA-A2.

Since Sur1 is a weak HLA-A2 binder, two analogue peptides designated Sur1L2 and Sur1M2, respectively in which a better anchor residue (leucine or methionine) replaced the native threonine at position 2 were synthesized. Both of these peptides bind with almost similar high affinity to HLA-A2 as the positive control (C₅₀=1 μM).

CTL Response to Survivin in CLL Patients

PBLs from four HLA-A2 positive CLL patients were stimulated once in vitro before examination in the ELISPOT assay. This procedure was chosen to extend the sensitivity of the ELISPOT. All of the above 10 survivin-derived peptides were included in the first line of experiments. Responses were detected to Sur1 and Sur9 and only data for these peptides are given in the figures. FIG. 1 shows CTL reactivity to Sur1 and Sur9 as determined in patient CLL1. Each spot represents a peptide reactive, INF-γ-producing cell. The average number of spots per peptide was calculated using a CCD scanning device and a computer system. Fifty-two Sur9 peptide specific spots (after subtraction of spots without added peptide) per 6×10⁵ were detected in the CLL1 patient (FIG. 1b). No response was detected to the weak HLA-A2 binding peptide Sur1, however the patient responded strongly to the strong HLA-A2 binding peptide analogue Sur1M2 (35 peptide specific spots per 10⁴ cells) (FIG. 2). No response was detected to the other strong HLA-A2 binding peptide analogue Sur1L2 in this patient (FIG. 2). Patient CLL2 responded strongly to Sur9 (128 peptide specific spots per 10⁵ cells) and weakly to Sur1 (22 peptide specific spots per 10⁵ cells) (FIG. 3). The response to the Sur1L2 analogue was only slightly increased relative to the natural epitope, whereas the patient responded similarly strongly to the Sur1M2 peptide as to the decamer peptide Sur9. In patient CLL3 a weak response to Sur9 was observed (FIG. 3). No response to Sur1 or the modified Sur1 peptides were observed in the patient. No survivin responses were detected in the last patient CLL4 (data not shown). PBLs from 6 healthy HLA-A2 positive controls were analyzed to investigate whether a response to survivin could be detected in healthy individuals. No response was observed in any of the controls to any of the survivin derived peptides.

CTL Response to Survivin in Melanoma Patients

T lymphocytes isolated from tumour infiltrated lymph nodes from HLA-A2 positive melanoma patients were examined. The freshly resected lymph node was minced into small fragments and crushed to release cells into culture. Cells were stimulated once with peptide in vitro before examination in the ELISPOT assay. Survivin specific T cells were detected in three of the six patients analyzed. A strong Sur9 response was detected in patient Mel2 and Mel3. A weaker response to the Surl peptide was also detected in these patients (FIG. 4). In Mel1 the response to the weakly binding peptide Sur1 was stronger than the response to the stronger HLA-A2 binder Sur9 (FIG. 4). No response was detected in the tumor-infiltrated lymph nodes from the last three melanoma patients (Mel4-6). PBLs from two of the survivin reacting patients, Mel1 and Mel2, and from two of the non-reacting patients, Mel4 and Mel5, were examined. No response could be detected to either Sur9 or Sur1 in PBLs from any of these patients (data not shown).

### EXAMPLE 2

Spontaneous Cytotoxic T-Cell Responses to Survivin-Ferived MHC class I-Restricted T-Cell Epitopes in Situ and Ex Vivo in Cancer Patients

Summary

Spontaneous cytotoxic T-cell responses to survivin derived MHC class I restricted T-cell epitopes were demonstrated in situ as well as ex vivo in breast cancer, leukemia, and melanoma patients. Moreover, survivin reactive T cells isolated by magnetic beads coated with MHC/peptide complexes were cytotoxic to HLA-matched tumours of different tissue types. Being a universal tumor antigen, survivin may serve as a widely applicable target for anti-cancer immunotherapy.

Materials and Methods

Construction of HLA-Peptide Complexes for T-Cell Staining and T-Cell Sorting

A recognition site for enzymatic biotinylation using biotin protein ligase (BirA) in fusion with the 5′-end of the extracellular domains of HLA A*0201 (residues 1-275) was expressed in E. coli BL21 (DE3). The recombinant protein was purified by size—(Sephadex G25, Pharmacia) and ion exchange (mono-Q, Pharmacia) chromatography from inclusion bodies solubilised in 8 M urea. The HLA A*0201 was folded in vitro by dilution in the presence of the modified survivin peptide Sur1M2 (LMLGEFLKL, SEQ ID NO:5) or the MAA peptide gp100154-163, and subsequently biotinylated as described previously (35, 36). After gel filtration on a Pharmacia Sephadex G25 column to remove unbound biotin, the protein was multimerised with streptavidin-FITC conjugated dextran molecules (kindly provided by L. Winther, DAKO, Denmark) to generate multivalent HLA-dextran compounds for immunohistochemistry. The HLA A*0201 construct was a kind gift of Dr. Mark M. Davis (Dept. of Microbiology and Immunology, Stanford University, Palo Alto, Calif.). Cell separation was performed as previously described (37). Briefly, 5×10⁶ streptavidin-conjugated magnetic beads (Dynal, Oslo, Norway) were washed twice in 200 μl cold PBS, 0.5 μg peptide/A*0201 monomers were added and the mixture incubated for 15 min. at room temperature. After two washes these beads were mixed with PBLs at a ratio of 1:10 and subsequently incubated for 1 h followed by a precipitation of bead-bound cells in a magnetic field. The precipitation step was repeated once.

Immunohistochemistry Stainings

For staining with FITC-conjugated multimeric peptide/MHC complexes, tissue sections were dried overnight and subsequently fixed in cold acetone for 5 min. All incubation steps were performed at room temperature and in the dark: (i) 45 min. of the primary antibody (1:100 diluted), (ii) Cy 3-conjugated goat anti-mouse (1:500 diluted; code 115-165-100, Jackson ImmunoResearch, obtained from Dianova, Hamburg, Germany) for 45 min. and finally (iii) the multimers for 75 min. Between each step the slides were washed two times for 10 min. in PBS/BSA 0.1%. The slides were mounted in vectashield and kept in the refrigerator until observed under the confocal microscope.

Cytotoxicity Assay

Conventional [51Cr]-release assays for CTL-mediated cytotoxicity were carried out as described in (13). Target cells were autologous EBV-transformed B-cell lines, the HLA-A2 positive breast cancer cell line MCF-7 (available at ATCC), the HLA-A2 positive melanoma cell line FM3 (38), the HLA-A2 negative breast cancer cell line BT-20 (available from ATCC) and the HLA-A2 negative melanoma cell line FM45 (38). All cancer cell lines expressed survivin as examined by RT-PCR (data not shown).

ELISPOT Assay

The ELISPOT assay was used to quantify peptide epitope-specific IFN-□ releasing effector cells and has been described previously (39). Briefly, nitrocellulose bottomed 96-well plates (MultiScreen MAIP N45, Millipore) were coated with an anti-IFN-γ antibody (1-D1K, Mabtech, Sweden) and non-specific binding was blocked using AIM V (GibcoBRL, Life Technologies Inc., Gaithersburg, Md., USA). Lymphocytes were added at different cell concentrations together with the specific peptides and T2 cells and incubated overnight at 37° C. Following two washes the biotinylated detection antibody (7-B6-1-Biotin, Mabtech) was added. Specific binding was visualised using alkaline phosphatase-avidin together with the respective substrate (GibcoBRL). The reaction was terminated upon the appearance of dark purple spots, which were quantitated using the Aiphalmager System (Alpha Innotech, San Leandro, Calif., USA). The peptides used for the ELISPOT were Sur1, Sur9 and the Sur1 analogue peptide Sur1M2 as described in Example 1.

Results

In Situ Staining of HLA-A2/Survivin Reactive T Cells

In Example 1 two survivin derived peptide epitopes recognized by T cells in leukemia and melanoma, i.e., Sur1 were identified. The weak binding affinity of Sur1 to HLA-A2 was improved substantially by replacing threonine at position 2 with a better anchor residue (methionine; Sur1M2). This measure enabled the construction of stable HLA-A2/peptide complexes. These complexes were multimerised using dextran molecules, which were conjugated with streptavidin and FITC. Multimerised MHC-complexes were used to stain acetone-fixed, frozen material. Using a confocal laser microscope, Sur1M2/HLA-A*0201 reactive CTLs could readily be detected in situ in the tumor microenvironment. We depicted such cells in the primary tumor and the sentinel lymph node of a stage III melanoma patient as well as in a primary breast cancer lesion (FIG. 5 and 6). To ensure the specificity of the staining, a series of negative controls (exemplified in FIG. 6, B and C) was carried out. Neither the use of peptide/HLA-dextran multimers with peptides derived from the melanoma differentiation antigen gp100 on the same tumour, nor Sur1M2/HLA-dextran multimers in case of a tumour sample obtained from an HLA-A2 negative donor resulted in a positive staining.

Isolated Survivin Reactive CTLs Lyse Tumour Cell Lines of Different Origin

To characterise the functional capacity of survivin-reactive CTLs, these cells were isolated by means of magnetic beads coated with HLA-A2/Sur1M2-complexes (36). A freshly resected melanoma infiltrated lymph node was minced into small fragments and crushed to release cells into culture. Cells were stimulated once with peptide in vitro prior to isolation. One day after isolation IL-2 was added, and on day 5 the capacity of these cells to kill tumour cells was tested either by ELISPOT or in standard 51Cr release assays. First, by means of ELISPOT analysis it was possible to establish that CTLs isolated using the modified Sur1M2/HLA-A2-complex also responded to the native Sur1 peptide (data not shown). Second, the cytotoxicity of the survivin reactive CTLs against the HLA-A2 positive melanoma cell-line FM3 (FIG. 7 A) and the HLA-A2 positive breast-cancer cell line MCF-7 (FIG. 7 B) was tested. The isolated T cells effectively lysed both HLA-A*0201 cell lines. In contrast, no cytotoxicity was observed against the HLA-A2 negative melanoma cell line FM45 (FIG. 7 A) or the HLA-A2 negative breast cancer cell line BT-20 (FIG. 7B).

Survivin Reactivity Measured in PBL by ELISPOT

The presence of survivin reactive T cells in PBLs from ten HLA-A2 positive breast cancer patients was examined by the ELISPOT. Before analysis, PBLs were stimulated once in vitro to extend the sensitivity of the assay. Reactivity to the following survivin peptides was examined: Sur1, Sur9 and Sur1M2. Survivin specific T cells were detected in six out of the ten HLA-A2 positive breast cancer patients. Representative examples are given in FIG. 8. In PBLs from two patients a response against Sur1 and the modified analogue Sur1M2, but not against Sur9 (FIG. 8, A and B) was detected, in three patients a response to Sur9 was detected, but not to Sur1 or Sur1M2 (FIG. 8 C), and one patient responded only to Sur1M2. In contrast, no survivin responses were detected in PBLs from 20 healthy HLA-A2 positive donors. Similarly, PBLs from fourteen HLA-A2 positive melanoma patients were examined. Survivin responses were present in seven of these patients (Table 2). Two patients responded to the Sur9 peptide, three to the Sur1M2 peptide, one to both Sur1 and SurM2, and one to all three peptides. In Example 1, T-cell response to survivin in 3 chronic lymphatic leukemia (CLL) patients was tested (Table 2; CLL1, CLL2, CLL3). These studies were extended using PBLs from three additional CLL patients. Notably, all patients produced a T-cell response to at least one survivin epitope (Table 2; CLL5, CLL6, CLL7). In addition, PBLs from one patient suffering from chronic myeloid leukemia (CML) was examined. In this patient, a response to all three peptides was identified (data not shown). The data are summarized in Table 2.

TABLE 2
Patients with survivin peptide-specific T lymphocytes in
PBLs as measured by ELISPOT

| | | | |
|---|---|---|---|
| Patient | Sur1 | Sur9 | Sur1M2 |

Melanoma a)

| | | | |
|---|---|---|---|
| P4 | — | — | 97 |
| P11 | — | — | 112 |
| P13 | — | — | 71 |
| P15 | 61 | — | 101 |
| P17 | — | 172 | — |
| P39 | — | 127 | — |
| P64 | 112 | 70 | 128 |

Breast cancer b)

| | | | |
|---|---|---|---|
| B1 | 122 | — | 208 |
| B2 | 67 | — | 72 |
| B3 | — | 54 | — |
| B4 | — | 45 | — |
| B5 | — | 19 | — |
| B6 | — | — | 24 |

CLL c)

| | | | |
|---|---|---|---|
| CLL1 | — | 27 | 320 |
| CLL2 | — | 39 | — |
| CLL3 | 23 | 127 | 122 |
| CLL5 | — | 100 | 124 |
| CLL6 | — | 121 | 360 |
| CLL7 | 68 | 132 | 174 |

a) Frequency of reactive cells per 10⁴; 14 patients examined.
b) Frequency of reactive cells per 10⁴; 10 patients examined.
c) Frequency of reactive cells per 10⁵; 7 patients examined.

### EXAMPLE 3

HLA-B35-Restricted Immune Responses to Survivin Derived Peptides in Cancer Patients

Summary

In this study, two survivin derived epitopes, which are restricted to HLA-B35 were identified and characterized. Specific T-cell reactivity against both of these epitopes was present in the peripheral blood from patients with different haematopoietic malignancies and melanoma. Substitutions of the C-terminal anchor residue improved the recognition by tumor infiltrating lymphocytes from melanoma patients. Furthermore, spontaneous cytotoxic T-cell responses to survivin in situ in a primary melanoma lesion was demonstrated. These epitopes extends the applicability of future vaccine strategies based on survivin peptides in relation to malignancies as well as the HLA profile of the patients involved.

In Examples 1 and 2, HLA-A2 restricted survivin-derived T-cell epitopes were studied. Since HLA-A2 is only expressed in about 30% of the Caucasian population (63), peptide epitopes restricted to other HLA class I molecules need to be identified to extend the fraction of patients that could be treated. In this study, two novel T-cell epitopes from survivin restricted to HLA-B35, which is expressed in 9% of the Caucasian population (63), were identified, and spontaneous immune responses to these survivin peptides were detected in patients with different haematopoietic malignancies and melanoma.

Materials and Methods

Patients

Peripheral vein blood samples from cancer patients were collected, PBLs were isolated using Lymphoprep separation, HLA-typed (Department of Clinical Immunology, University Hospital, Copenhagen) and frozen in FCS with 10% DMSO. Ten HLA-B35 positive patients were selected for further analysis. These patients suffered from melanoma, CLL, follicular lymphoma (FL), diffuse large B-cell lymphomas (DLBCL) and Multiple Myeloma (MM), respectively. At the time blood samples were collected patients had not been medically treated within the previous four months. Additionally, tumor-infiltrating lymphocytes (TIL) isolated from lymph nodes were collected from three of the melanoma patients and frozen in FCS with 10% DMSO.

Peptides

Seven synthetic survivin derived peptides were used in this study: Sur6-14, Sur11-19, Sur34-43, Sur46-54, Sur51-59, Sur46Y9, Sur51Y9, and one EBV-derived peptide, EBNA3A 457-466 (63). All peptides were obtained from Research Genetics (Huntsville, Ala.) and provided at >90% purity, as verified by HPLC and MC analyses. The peptides are listed in Table 3 below.

TABLE 3
HLA-B35 binding of survivin derived peptides

| | | | | |
|---|---|---|---|---|
| Name | Protein and position | Sequence | SEQ ID NO: | C₅₀ (μM) |

| | | | | |
|---|---|---|---|---|
| Sur6-14 | Survivin₆₋₁₄ | LPPAWQPFL | 18 | >100 |
| Sur11-19 | Survivin₁₁₋₁₉ | QPFLKDHRI | 19 | Not binding |
| Sur34-43 | Survivin₃₄₋₄₃ | TPERMAEAGF | 20 | >100 |
| Sur46-54 | Survivin₄₆₋₅₄ | CPTENEPDL | 6 | 20 |
| Sur51-59 | Survivin₅₁₋₅₉ | EPDLAQCFF | 7 | 13 |
| Sur46Y9 | Modified peptide | CPTENEPDY | 8 | 4 |
| Sur51Y9 | Modified peptide | EPDLAQCFY | 9 | 1.5 |
| C24 | EBNA3A₄₅₈₋₄₆₆ | YPLHEQHQM | 21 | 0.8 |

Assembly Assay for Peptide Binding to MHC Class I Molecules

The assembly assay described in Examples 1 and 2 was used to measure binding affinity of the synthetic peptides to HLA-B35 molecules metabolically labeled with [S35]methionine. Briefly, the assay is based on peptide-mediated stabilization of empty HLA molecules released, upon cell lysis, from the TAP deficient cell line T2, stably transfected with HLA-B35 (kindly provided by Dr J. Haurum, Symphogen ApS, Lyngby, Denmark). Stably folded HLA-molecules were immunoprecipitated using the conformation-dependent mAb W6/32. The HLA molecules were separated by IEF electrophoresis, gels were exposed to phosphorimager screens (Imaging plate, FUJI photo film Co., LTD., Japan), analyzed and the amount of correctly folded HLA molecules were quantified using ImageGauge phosphorimager software (FUJI photo film Co., LTD., Japan).

Antigen Stimulation of PBLs

To extend the sensitivity of the ELISPOT assay, lymphocytes were stimulated once in vitro with peptide prior to analysis (14, 15). PBLs or TILs were thawed and stimulated with 50 μM of the individual peptide epitopes in 96-well plates for 2 h at 26° C. (5×10⁵-10⁶ cells per peptide), and pooled for further 10 days of culture at 37° C. in x-vivo with 5% human serum (HS), in 24 well plates (Nunc, Roskilde, Denmark), with 2×10⁶ cells per well. At the second day of incubation 40 μg/ml IL-2 (Apodan A/S, Denmark) were added. At day 10, the cultured cells were tested for reactivity in the ELISPOT assay.

The ELISPOT Assay

The ELISPOT assay used to quantify peptide specific, IFN-γ releasing effector cells in PBLs or TILs collected from cancer patients was performed as described in Example 1. Briefly, nitrocellulose-bottomed 96-well plates (MultiScreen MAIP N45; Millipore, Hedehusene, Denmark) were coated with mAb against human IFN-γ, 7.5 μg/ml (1-D1K; Mabtech, Nacka, Sweden). Wells were washed and blocked in x-vivo (x-vivo 15TM BioWhittacker, Molecular Applications Aps, Denmark) and cells were added in duplicates at different concentrations. For antigen presentation, 10⁴ T2-B35 cells, with and without 10 μM peptide, were added per well. Plates were incubated overnight, the cells discarded, and wells washed prior to addition of biotinylated secondary antibody (7-B6-1-Biotin; Mabtech). Plates were incubated 2 h at room temperature, washed and avidin-alkaline phosphatase conjugate was added (AP-Avidin; Calbiochem, Life Technologies, Inc.). After 1 h of incubation at room temperature, the enzyme substrate nitroblue tetrazolium/5-bromo-4-chloro-3-indolyl phosphate (Code No.K0598, DakoCytomation Norden A/S) was added, and dark purple spots emerged in 3-7 min. The reaction was terminated by washing with tap water. Spots were counted using the Alpha Imager System (Alpha Innotech, San Leandro, Calif.), and the frequency of peptide specific T cells were calculated from the number of spot forming cells.

All assays were performed in duplicates for each peptide antigen, and lymphocytes cultured in the same well, were tested in equal cell numbers with and without peptide, to measure the number of peptide specific cells in the culture.

Maturation of Dendritic Cells (DCs)

Adherent cells were isolated from PBLs after 2 h of culture. These were cultured for 10 additional days in RPMI 1640 (GibcoTM Invitrogen corporation, UK) with 10% FCS. 800 ng/ml GM-CSF (PreproTech, London, UK) and 40 ng/ml IL-4 (PreproTech) were added every third day. At day 10, DCs were matured for 24 h by adding 50 ng/ml TNF-α (PreproTech). After maturation, DCs were released and pulsed with 20 μM peptide in the presence of 3 μg/ml β2-microglobulin for 2 h at 26° C.

Isolation of Peptide Specific T Cells

Antigen specific cells were isolated using sur51Y9/HLA-B35-coated magnetic beads as described in Example 2. Biotinylated monomers of HLA-B35 with sur51Y9 (obtained from ProImmune, Oxford, UK) were coupled to streptavidin coated magnetic beads (Dynabeads M-280, Dynal A/S, Oslo, Norway) by incubating 2.5 μg monomers with 5×10⁶ beads in 40 μl PBS for 20 min. at room temperature. The magnetic complexes were washed three times in PBS, using a magnetic device (Dynal A/S, Oslo, Norway) and subsequently mixed with PBLs at a ratio of 1:10 in PBS with 5% BSA, and rotated very gently for 1 h. Antigen specific CD8⁺ T cells associating with the magnetic complexes were gently washed two or three times. Isolated cells were resuspended several times in x-vivo supplemented with 5% human serum and incubated for 2 h before the magnetic beads were released and removed from the cell suspension. The isolated antigen specific CD8⁺ T cells were used in ELISPOT assay to analyze the cross-reactivity between the native and modified peptide.

TCR Clonotype Mapping by Denaturing Gradient Gel Electrophoresis (DGGE)

DGGE clonotype mapping of the human TCR BV regions 1-24 has been described in details (66). Briefly, RNA was isolated using the Purescript Isolation Kit (Gentra Systems Inc. Minn.) and transcribed cDNA was amplified by PCR using primers for the variable regions of the TCR beta chains in conjunction with a common constant region primer. The computer program MELT87 was used to ensure that the amplified DNA molecules were suited for DGGE analysis provided a 50 bp GC-rich sequence (GC-clamp) was attached to the 5′-end of the constant region primer. DGGE analysis was done in 6% polyacrylamide gels containing a gradient of urea and formamide from 20% to 80%. Electrophoresis was performed at 160 V for 4.5 hours in lx TAE buffer at a constant temperature of 54° C.

Immunohistochemistry Stainings

Multimerised peptide/HLA complexes were used to identify antigen specific T cells in situ in tumor lesions of cancer patients using the procedure described in Example 2. Biotinylated sur51Y9/HLA-B35 monomer was supplied by Proimmune limited, Oxford, UK. The biotinylated monomers of sur51Y9/HLA-B35 were multimerised with streptavidin-FITC-conjugated dextran molecules (kindly provided by L. Winther, DAKO, Glostrup, Denmark) to generate multivalent HLA-dextran compounds for immunohistochemistry. Tissue sections were dried overnight and subsequently fixed in cold acetone for 5 min. All the incubation steps were performed in the dark at room temperature: (a) 45 min of the primary antibody (1:100 diluted) (b) Cy 3-conjugated goat-anti-mouse antibody (1:500 diluted; code 115-165-100; Jackson ImmunoResearch, obtained from Dianova, Hamburg, Germany) for 45 min; and finally (c) the multimers for 75 min. Between each step, the slides were washed two times for 10 min in PBS/BSA 0.1%. The slides were mounted in vectashield and kept in the refrigerator until observed under the confocal microscope (Leica).

Results

Identification of HLA-B35 Binding Survivin Derived Peptides

The amino acid sequence of survivin was screened for nonameric and decameric peptides with anchor residues, according to the peptide-binding motif of HLA-B35 (67). Five peptides were selected containing proline as the N-terminal anchor in position 2 and phenyl-alanine, leucine, isoleucine or tyrosine as C-terminal anchor residues (Table 3). Assembly assay revealed two peptides, sur51-59 (EPDLAQCFF, SEQ ID NO:7) and sur46-54 (CPTENEPDL, SEQ ID NO:6) that were able to stabilise HLA-B35 efficiently. Additionally, two peptides, sur34-43 (TPERMAEAGF, SEQ ID NO:20) and sur6-14 (LPPAWQPFL, SEQ ID NO: 18) showed a weak stabilization, whereas the remaining peptide did not stabilize HLA-B35 at all. The peptide concentration required for half maximal recovery of HLA-B35 (C50) was estimated at 13 μM for sur51-59 and 20 μM for sur46-54. In comparison, the positive control-epitope C24 from EBNA3A458-466 (YPLHEQHQM, SEQ ID NO:21) had an estimated C₅₀ value of 0,8 μM.

To enhance the binding affinity of sur46-54 and sur51-59 the C-terminal amino acid was replaced with tyrosine, a better anchor residue (67). The recovery of HLA-B35 mediated by the modified peptides was analyzed in the assembly assay, and C₅₀ values were estimated at 1.5 μM for sur51Y9 and 4 μM for sur46Y9 (FIG. 9).

Spontaneous Immune Responses Against Native Peptide Epitopes

Initially, five patients were analyzed for spontaneous immune responses to the four native HLA-B35 binding peptides sur51-59, sur46-54, sur34-43 and sur6-14. These five patients had different haematopoietic malignancies: HEM8 and HEM18 suffered from MM, HEM12 from FL, HEM9 had DLBCL, and CLL5 had CLL.

INF-γ ELISPOT assays were performed on PBLs after 10 days of in vitro stimulation to detect peptide precursor CTLs. Spontaneous immune responses were detected against two of the native HLA-B35 binding peptides, sur51-59 and sur46-54. Two patients, HEM12 and CLL5 showed a response to both sur51-59 and sur46-54, whereas HEM8 only showed a response to sur51-59 (FIG. 10A-C). No response could be detected in the two remaining patients, HEM9 and HEM18, and no response could be detected to the poorly binding peptides sur34-46 and sur6-14 in any patients.

An alternative approach to in vitro stimulation was used in patient HEM12, i.e. PBLs were co-cultured with matured autologous dendritic cells pulsed with sur51-59 to stimulate a CTL response in vitro. PBLs from this culture showed strong reactivity towards sur51-59 in ELISPOT (FIG. 10D).

Increased Recognition of Modified Peptides

As described above, peptide modifications to enhance the HLA-B35 affinity resulted in a 5-10-fold higher affinity for HLA-B35 relative to the native peptides. A group of five melanoma patients were analyzed for spontaneous immune responses to both the native and modified peptides by means of ELISPOT assay. PBL samples were analyzed after in vitro stimulation, whereas TIL samples were analyzed directly. Spontaneous immune responses were observed in either PBLs or TILs from three of the five patients. FM25 showed reactivity against sur51-59 and sur51Y9 in both PBL and TIL samples (FIG. 11A). FM45 responded only to the modified peptide sur51Y9, with a strong response detectable in TILs. No PBLs were available from this patient (FIG. 11A). FM74 showed a strong response to sur46Y9 in TIL, but no response to the native peptide was detectable (FIG. 11B). A weak response to sur46Y9 was also observed in PBLs from FM74 (data not shown).

Cross-Reactivity Between the Native and Modified Peptide

The high affinity of sur51Y9 to HLA-B35 enables the production of stable monomers of HLA-B35 with sur51Y9. Having established the presence of survivin reactive T lymphocytes in tumor infiltrated lymph nodes and PBLs from different cancer patients, magnetic beads were coated with such HLA-B35/Sur51Y9-complexes and these were used to isolate survivin peptide reactive T lymphocytes from PBL from patient CLL5. This patient showed a strong response to sur51-59. Beads were tightly bound to the cell surface of the specific cells, as visualized by microscopy (data not shown), permitting precipitation of antigen specific cells by a magnetic field. The isolated sur51Y9 specific cells responded strongly to sur51-59, (FIG. 11C), whereas no response could be detected in the remaining PBLs (data not shown). The isolation was analyzed by the RT-PCR/DGGE based TCR clonotype mapping. This technique allows the analysis for T-cell clonality in complex cell populations, even if only small numbers of cells are available. These analyses showed that 8 distinct clones were isolated (data not shown).

Antigen Specific T Cells Present in Situ in a Melanoma Lesions

Sur51Y9/HLA-B35 monomers were multimerised using dextran molecules conjugated with streptavidin and FITC. Multimerised MHC-complexes were used to stain acetone-fixed, frozen material using the procedure described in Example 2. Antigen specific cells were visualized using a confocal laser microscope. Sections of primary melanoma from three patients were analyzed, and Sur51Y9/HLA-B35-reactive CTLs could readily be detected in situ in the tumor microenvironment in one of the patients (FIG. 12). Co-staining with a mAb against granzyme B showed that these survivin specific CTLs released granzyme B, exerting cytotoxic activity (FIG. 12) HLA-B35 negative melanoma patients were used as controls.

### EXAMPLE 4

HLA Allele-Restricted Immune Responses to Survivin Derived Peptides in Cancer Patients

A range of survivin derived peptides comprising 9-11 amino acid residues were tested for binding to the following HLA alleles: HLA-A1, HLA-A3, HLA-A11 and HLA-B7 using the assembly assay for peptide binding to MHC class I molecules described in the preceding examples. In addition, several of the peptides were tested for their capacity to elicit a CTL immune response using the ELISPOT assay as also described above.

A summary of the results are given in the below Table 4:

TABLE 4
C₅₀ and ELISPOT data for selected survivin derived peptides

| HLA | Peptide | | | | | SEQ ID | ELISPOT |
|---|---|---|---|---|---|---|---|
| allele | length | Position | Sequence | C₅₀ (μM) | Remarks | NO: | Results |
| HLA-A1 | 9mer | Sur14-22 | LKDHRISTF | NB | | 22 | |
| | | Sur51-59 | EPDLAQCFF | NB | | 7 | |
| | | Sur38-46 | MAEAGFIHC | | Not analyzed | 23 | |
| | | Sur93-101 | FEELTLGEF | >100 | | 24 | |
| | 10mer | Sur34-43 | TPERMAEAGF | NB | | 20 | |
| | | Sur47-56 | PTENEPDLAQ | | Not analyzed | 25 | |
| | | Sur49-58 | ENEPDLAQCF | NB | | 26 | |
| | | Sur92-101 | QFEELTLGEF | 2 | | 27 | |
| | Control | C1 | VSDGGPNLY | 0.8 | | 28 | |
| | peptide | | | | | | |
| | Modified | sur14Y9 | LKDHRISTY | NB | | 29 | |
| | peptides | | | | | | |
| | | sur51Y9 | EPDLAQCFY | | Weak | 9 | |
| | | | | | binding | | |
| | | sur93Y9 | FEELTLGEY | NB | | 30 | |
| | | sur92Y9 | QFEELTLGEY | NB | | 31 | |
| | | sur34Y9 | TPERMAEAGY | NB | | 32 | |
| | | sur49Y9 | ENEPDLAQCY | NB | | 33 | |
| | | Sur92T2 | QTEELTLGEF | | Not analyzed | 34 | |
| | | Sur92S2 | QSEELTLGEF | | Not analyzed | 35 | |
| | | Sur93T2 | FTELTLGEF | | Not analyzed | 36 | |
| | | Sur93S2 | FSELTLGEF | | Not analyzed | 37 | |
| | | Sur38Y9 | MAEAGFIHY | | Not analyzed | 38 | |
| | | Sur46Y10 | PTENEPDLAY | | Not analyzed | 39 | |
| HLA-A3 | 9mer | Sur5-13 | TLPPAWQPF | NB | | 40 | |
| | | Sur53-61 | DLAQCFFCF | NB | | 41 | |
| | | Sur54-62 | LAQCFFCFK | NB | | 42 | |
| | | Sur95-103 | ELTLGEFLK | >100 | | 43 | |
| | | Sur112-120 | KIAKETNNK | 2 | | 44 | i |
| | 10mer | Sur13-22 | FLKDHRISTF | NB | | 45 | |
| | | Sur18-26 | RISTFKNWPF | NB | | 46 | |
| | | Sur53-62 | DLAQCFFCFK | 100 | | 47 | ii |
| | | Sur84-92 | CAFLSVKKQF | NB | | 48 | |
| | | Sur101-120 | FLKLDRERAK | NB | | 49 | |
| | | Sur103-112 | KLDRERAKNK | NB | | 50 | |
| | | Sur112-121 | KIAKETNNKK | 1 | | 51 | |
| | | Sur113-125 | IAKETNNKKK | NB | | 52 | |
| | Control | C3 | ILRGSVAHK | 0.1-0.3 | | 53 | |
| | peptide | | | | | | |
| | Modified | Sur5K9 | TLPPAWQPK | 2 | | 54 | |
| | peptides | | | | | | |
| | | Sur53K9 | DLAQCFFCK | NB | | 55 | |
| | | Sur54L2 | LLQCFFCFK | 1 | | 56 | |
| | | Sur13K9 | FLKDHRISTK | NB | | 57 | |
| | | Sur18K9 | RISTFKNWPK | 0.1 | | 58 | iii |
| | | Sur113L2 | ILKETNNKKK | | Weak | 59 | |
| | | | | | binding | | |
| | | SurEx3-A3-1 | TIRRKNLRK | 0.5 | | 60 | iv |
| | | SurEx3-A3-2 | PTIRRKNLRK | NB | | 61 | |
| | | Sur2b-A3-1 | RITREEHKK | NB | | 62 | |
| HLA- | 9mer | Sur5-13 | TLPPAWQPF | NB | | 40 | |
| A11 | | | | | | | |
| | | Sur53-61 | DLAQCFFCF | NB | | 41 | |
| | | Sur54-62 | LAQCFFCFK | 0.4 | | 42 | v |
| | | Sur95-103 | ELTLGEFLK | NB | | 43 | |
| | | Sur112-120 | KIAKETNNK | 1 | | 44 | |
| | 10mer | Sur13-22 | FLKDHRISTF | NB | | 45 | |
| | | Sur18-26 | RISTFKNWPF | NB | | 46 | |
| | | Sur53-62 | DLAQCFFCFK | 5 | | 47 | vi |
| | | Sur84-92 | CAFLSVKKQF | NB | | 48 | |
| | | Sur101-120 | FLKLDRERAK | NB | | 49 | |
| | | Sur103-112 | KLDRERAKNK | NB | | 50 | |
| | | Sur112-121 | KIAKETNNKK | >100 | | 51 | vii |
| | | Sur113-125 | IAKETNNKKK | NB | | 52 | |
| | Control | C4 | AVFDRKSDAK | 0.2 | | 63 | |
| | peptide | | | | | | |
| HLA-B7 | 9mer | Sur6-14 | LPPAWQPFL | >100 | | 18 | viii |
| | | Sur11-19 | QPFLKDHRI | >100 | | 19 | |
| | | Sur46-54 | CPTENEPDL | NB | | 6 | |
| | | Sur51-59 | EPDLAQCFF | NB | | 7 | |
| | 10mer | Sur34-43 | TPERMAEAGF | NB | | 20 | |
| | Control | C6 | QPRAPIRPI | 0.1 | | 64 | |
| | peptides | | | | | | |
| | | C7 | RPPIFIRRL | 0.5 | | 65 | |

i A weak response was observed in one lymphoma patient, HEM34.
ii Weak responses were detected in 3 lymphoma patients (HEM9, 11, 34).
iii Weak responses were observed in 4 melanoma patients (TIL37, FM-TIL78, FM-TIL95, pt.76).
iv A weak response was observed in a melanoma patient (FM-TIL95).
v Weak responses were detected in two MM patients (HEM47, 48).
vi A weak response was observed in a melanoma patient (PM6), in PBL and metastatic lymph-node suspension.
vii A response was observed in a melanoma patient (PM6), most evident in metastatic lymph-node suspension, and weaker in the TIL from primary tumor and PBL.
viii A response was observed in a CLL patient (CLL9), and a weaker response was observed in a lymphoma patient (HEM21).

### EXAMPLE 5

Therapeutic Trial Procedures Using Survivin Derived Peptides as Immunogens

Stage IV metastatic melanoma patients with progressive disease were entered into a Dendritic cell (DC)-based vaccination trial after having failed to respond to chemotherapy. All patients provided informed consent to participate in the experimental vaccination and to donate blood for immunological monitoring. Serological HLA typing revealed that the patients were HLA-A2. Dendritic cells (DC) were pulsed with Sur1M2 peptide and 5×10⁶ cells were administered subcutaneously at day 1 and 14, subsequently every 4 weeks, additional leukapheresis after 5 vaccinations.

The generation of DCs for clinical use and quality control was performed as described in Thurner et al. J. Immunol. Methods 223:1 (1999). All vaccine preparations were highly enriched in respect of mature DCs with >90% showing a characteristic phenotype by flow cytometry (HLA-DR+++, CD86+++, CD40+, CD25+, CD14−). More than 80% of the cells expressed the CD83 antigen as marker for mature DCs. The peptide Sur1M2 (LMLGEFLKL) (SEQ ID NO: 10) used in the vaccination trial was synthesized at a GMP quality by Clinalfa (purity >98%).

Immunological Responses

The presence of survivin reactive T cells in PBLs from 4 vaccinated HLA-A2 positive melanoma patients was examined by the ELISPOT procedure as described above. Prior to analysis, PBLs were stimulated once in vitro to extend the sensitivity of the assay. Reactivity to the following survivin peptides was examined: (i) Sur1 (SEQ ID NO:10); (ii) Sur9 (SEQ ID NO:3); and (iii) Sur1M2 (SEQ ID NO:5).

Patient RW did not host a survivin response prior to vaccination (Table 5). However, after 5 vaccinations a strong response against all three peptides was detected. Similarly, in patient KN a response against all three peptides could be detected after 5 vaccinations. However, these responses were not detectable after 10 vaccinations. Additionally, we detected a response against peptide Sur1M2 in patient WW and GB after, but not prior, to vaccination (Table 5).

The ELISPOT procedure offers a unique possibility to measure the number of antigen-specific T cells in vaccination trials. It has previously been used for the measurement of reactive T cells in autoimmune disease or infection, and recently, to give a reliable estimate of the proportion of antigen-reactive T cells in cancer patients. Using the ELISPOT analysis we demonstrated the induction of a survivin peptide response in all patients subsequent to vaccination with DCs.

TABLE 5

Summary of vaccination trials: Survivin
specific cells in PBLs from sur1m2-vaccinated
patients

| | |
|---|---|
| No. of | Peptide specific cells per 10⁵ PBL |

| | | | | |
|---|---|---|---|---|
| Pa- | vacci- | Sur1M2 | Sur1 | Sur9 |
| tient | nations | (LMLGEFLKL) | (LTLGEFLKL) | (ELTLGEFLKL) |

| | | | | |
|---|---|---|---|---|
| RW | Before | 5 | 6 | 5 |
| | 5 | 110 | 131 | 130 |
| KN | Before | 4 | 8 | 6 |
| | 3 | 12 | 12 | 10 |
| | 5 | 50 | 100 | 50 |
| | 10 | 0 | 1 | 0 |
| WW | Before | 0 | NOT DONE | NOT DONE |
| | 5 | 64 | NOT DONE | NOT DONE |
| GB | Before | 1 | NOT DONE | NOT DONE |
| | 5 | 150 | NOT DONE | NOT DONE |

### REFERENCES

1. Van den Eynde, B. J. and Boon, T. Tumor antigens recognized by T lymphocytes. Int. J. Clin. Lab. Res., 27: 81-86, 1997.

2. Rosenberg, S. A. Development of cancer immunotherapies based on identification of the genes encoding cancer regression antigens. J. Natl. Cancer Inst., 20;88: 1635-1644, 1996.

3. Marchand, M., van Baren, N., Weynants, P., Brichard, V., Dreno, B., Tessier, M. H., Rankin, E., Parmiani, G., Arienti, F., Humblet, Y., Bourlond, A., Vanwijck, R., Lienard, D., Beauduin, M., Dietrich, P. Y., Russo, V., Kerger, J., Masucci, G., Jager, E., De Greve, J., Atzpodien, J., Brasseur, F., Coulie, P. G., van der Bruggen, P., and Boon, T. Tumor regressions observed in patients with metastatic melanoma treated with an antigenic peptide encoded by gene MAGE-3 and presented by HLA-A1. Int. J. Cancer, 80: 219-230, 1999.

4. Brossart, P., Stuhler, G., Flad, T., Stevanovic, S., Rammensee, H. G., Kanz, L., and Brugger, W. Her-2/neu-derived peptides are tumor-associated antigens expressed by human renal cell and colon carcinoma lines and are recognized by in vitro induced specific cytotoxic T lymphocytes. Cancer Res., 58: 732-736, 1998.

5. Brossart, P., Heinrich, K. S., Stuhler, G., Behnke, L., Reichardt, V. L., Stevanovic, S., Muhm, A., Rammensee, H. G., Kanz, L., and Brugger, W. Identification of HLA-A2-restricted T-cell epitopes derived from the MUC1 tumor antigen for broadly applicable vaccine therapies. Blood, 93: 4309-4317, 1999.

6. Vonderheide, R. H., Hahn, W. C., Schultze, J. L., and Nadler, L. M. The telomerase catalytic subunit is a widely expressed tumor-associated antigen recognized by cytotoxic T lymphocytes. Immunity, 10: 673-679, 1999.

7. LaCasse, E. C., Baird, S., Korneluk, R. G., and MacKenzie, A. E. The inhibitors of apoptosis (IAPs) and their emerging role in cancer. Oncogene, 17: 3247-3259, 1998.

8. Altieri, D. C., Marchisio, P. C., and Marchisio, C. Survivin apoptosis: an interloper between cell death and cell proliferation in cancer. Lab Invest, 79: 1327-1333, 1999.

9. Ambrosini, G., Adida, C., Sirugo, G., and Altieri, D. C. Induction of apoptosis and inhibition of cell proliferation by survivin gene targeting. J. Biol. Chem., 273: 11177-11182, 1998.

10. Grossman, D., McNiff, J. M., Li, F., and Altieri, D. C. Expression and targeting of the apoptosis inhibitor, survivin, in human melanoma. J. Invest Dermatol., 113: 1076-1081, 1999.

11. Grossman, D., McNiff, J. M., Li, F., and Altieri, D. C. Expression of the apoptosis inhibitor, survivin, in nonmelanoma skin cancer and gene targeting in a keratinocyte cell line. Lab Invest, 79: 1121-1126, 1999.

12. Andersen, M. H., Sondergaard, I., Zeuthen, J., Elliott, T., and Haurum, J. S. An assay for peptide binding to HLA-Cw*0102. Tissue Antigens., 54: 185-190, 1999.

13. Andersen, M. H., Bonfill, J. E., Neisig, A., Arsequell, G., ndergaard, I., Neefjes, J., Zeuthen, J., Elliott, T., and Haurum, J. S. Phosphorylated Peptides Can Be Transported by TAP Molecules, Presented by Class I MHC Molecules, and Recognized by Phosphopeptide-Specific CTL. J. Immunol., 163: 3812-3818, 1999.

14. McCutcheon, M., Wehner, N., Wensky, A., Kushner, M., Doan, S., Hsiao, L., Calabresi, P., Ha, T., Tran, T. V., Tate, K. M., Winkelhake, J., and Spack, E. G. A sensitive ELISPOT assay to detect low-frequency human T lymphocytes. J. Immunol. Methods, 210: 149-166, 1997.

15. Pass, H. A., Schwarz, S. L., Wunderlich, J. R., and Rosenberg, S. A. Immunization of patients with melanoma peptide vaccines: immunologic assessment using the ELISPOT assay. Cancer J. Sci. Am., 4: 316-323, 1998.

16. Berke, Z., Andersen, M. H., Pedersen, M., Fugger, L., Zeuthen, J., and Haurum, J. S. Peptides spanning the junctional region of both the abl/bcr and the bcr/abl fusion proteins bind common HLA class I molecules. Leukemia, 14: 419-426, 2000.

17. Falk, K., Rotzschke, O., Stevanovic, S., Jung, G., and Rammensee, H. G. Allele-specific motifs revealed by sequencing of self-peptides eluted from MHC molecules. Nature, 351: 290-296, 1991.

18. Cornelison, T. L. Human papillomavirus genotype 16 vaccines for cervical cancer prophylaxis and treatment. Curr. Opin. Oncol., 12: 466-473, 2000.

19. Lee, S. P., Chan, A. T., Cheung, S. T., Thomas, W. A., CroomCarter, D., Dawson, C. W., Tsai, C. H., Leung, S. F., Johnson, P. J., and Huang, D. P. CTL control of EBV in nasopharyngeal carcinoma (NPC): EBV-specific CTL responses in the blood and tumors of NPC patients and the antigen-processing function of the tumor cells. J. Immunol., 165: 573-582, 2000.

20. Swana, H. S., Grossman, D., Anthony, J. N., Weiss, R. M., and Altieri, D. C. Tumor content of the antiapoptosis molecule survivin and recurrence of bladder cancer. N. Engl. J. Med., 341: 452-453, 1999.

21. Salgaller, M. L., Afshar, A., Marincola, F. M., Rivoltini, L., Kawakami, Y., and Rosenberg, S. A. Recognition of multiple epitopes in the human melanoma antigen gp100 by peripheral blood lymphocytes stimulated in vitro with synthetic peptides. Cancer Res., 55: 4972-4979, 1995.

22. Salgaller, M. L., Marincola, F. M., Cormier, J. N., and Rosenberg, S. A. Immunization against epitopes in the human melanoma antigen gp100 following patient immunization with synthetic peptides. Cancer Res., 56: 4749-4757, 1996.

23. Valmori, D., Fonteneau, J. F., Lizana, C. M., Gervois, N., Lienard, D., Rimoldi, D., Jongeneel, V., Jotereau, F., Cerottini, J. C., and Romero, P. Enhanced generation of specific tumor-reactive CTL in vitro by selected Melan-A/MART-1 immunodominant peptide analogues. J. Immunol., 160: 1750-1758, 1998.

24. Pardoll, D. M. Cancer vaccines. Nat. Med., 4: 525-531, 1998.

25. Kugler, A., Stuhler, G., Walden, P., Zoller, G., Zobywalski, A., Brossart, P., Trefzer, U., Ullrich, S., Muller, C. A., Becker, V., Gross, A. J., Hemmerlein, B., Kanz, L., Muller, G. A., and Ringert, R. H. Regression of human metastatic renal cell carcinoma after vaccination with tumor cell-dendritic cell hybrids. Nat. Med., 6: 332-336, 2000.

26. Becker, J. C., Guldberg, P., Zeuthen, J., Bröcker, E. B., and thor Straten, P. Accumulation of identical T cells in melanoma and vitiligo-like leukoderma. J. Invest. Dermatol., 113: 1033-1038, 1999.

27. Rohayem, J., Diestelkoetter, P., Weigle, B., Oehmichen, A., Schmitz, M., Mehlhorn, J., Conrad, K., and Rieber, E. P. Antibody response to the tumor-associated inhibitor of apoptosis protein survivin in cancer patients. Cancer Res. 2000. Apr. 1.;60.(7.):1815.-7, 60: 1815-1817.

28. Adida, C., Haioun, C., Gaulard, P., Lepage, E., Morel, P., Briere, J., Dombret, H., Reyes, F., Diebold, J., Gisselbrecht, C., Salles, G., Altieri, D. C., and Molina, T. J. Prognostic significance of survivin expression in diffuse large B-cell lymphomas. Blood, 96: 1921-1925, 2000.

29. Islam, A., Kageyama, H., Takada, N., Kawamoto, T., Takayasu, H., Isogai, E., Ohira, M., Hashizume, K., Kobayashi, H., Kaneko, Y., and Nakagawara, A. High expression of Survivin, mapped to 17q25, is significantly associated with poor prognostic factors and promotes cell survival in human neuroblastoma. Oncogene, 19: 617-623, 2000.

30. Kawasaki, H., Altieri, D. C., Lu, C. D., Toyoda, M., Tenjo, T., and Tanigawa, N. Inhibition of apoptosis by survivin predicts shorter survival rates in colorectal cancer. Cancer Res., 58: 5071-5074, 1998.

31. Schmitz, M., Diestelkoetter, P., Weigle, B., Schmachtenberg, F., Stevanovic, S., Ockert, D., Rammensee, H. G., and Rieber, E. P. Generation of survivin-specific CD8+ T effector cells by dendritic cells pulsed with protein or selected peptides. Cancer Res., 60: 4845-4849, 2000.

32. Andersen, M. H., Pedersen, L. O., Becker, J. C., and thor Straten, P. Identification of a Cytotoxic T Lymphocyte Response to the Apoptose Inhibitor Protein Survivin in Cancer Patients. Cancer Res., 61: 869-872, 2001.

33. Lee, K. H., Panelli, M. C., Kim, C. J., Riker, A. I., Bettinotti, M. P., Roden, M. M., Fetsch, P., Abati, A., Rosenberg, S. A., and Marincola, F. M. Functional dissociation between local and systemic immune response during anti-melanoma peptide vaccination. J. Immunol., 161: 4183-4194, 1998.

34. Rosenberg, S. A., Yang, J. C., Schwartzentruber, D. J., Hwu, P., Marincola, F. M., Topalian, S. L., Restifo, N. P., Dudley, M. E., Schwarz, S. L., Spiess, P. J., Wunderlich, J. R., Parkhurst, M. R., Kawakami, Y., Seipp, C. A., Einhorn, J. H., and White, D. E. Immunologic and therapeutic evaluation of a synthetic peptide vaccine for the treatment of patients with metastatic melanoma. Nat. Med., 4: 321-327, 1998.

35. Altman, J. D., Moss, P. A., Goulder, P. J. R., Barouch, D. H., McHeyzer Williams, M. G., Bell, J. I., McMichael, A. J., and Davis, M. M. Phenotypic analysis of antigen-specific T lymphocytes. Science, 274: 94-96, 1996.

36. Schrama, D., Andersen, M. H., Terheyden, P., Schroder, L., Pedersen, L. O., thor Straten, P., and Becker, J. C. Oligoclonal T-Cell Receptor Usage Of Melanocyte Differentiation Antigen-reactive T Cells in Stage IV Melanoma Patients. Cancer Res., 61: 493-496, 2001.

37. Luxembourg, A. T., Borrow, P., Teyton, L., Brunmark, A. B., Peterson, P. A., and Jackson, M. R. Biomagnetic isolation of antigen-specific CD8+ T cells usable in immunotherapy. Nat. Biotechnol., 16: 281-285, 1998.

38. Kirkin, A. F., Reichert Petersen, T., Olsen, A. C., Li, L., thor Straten, P., and Zeuthen, J. Generation of human-melanoma specific T lymphocyte clones defining novel cytolytic targets with panels of newly established melanoma cell lines. Cancer Immunol. Immunother., 41: 71-81, 1995.

39. Scheibenbogen, C., Lee, K. H., Mayer, S., Stevanovic, S., Moebius, U., Herr, W., Rammensee, H. G., and Keilholz, U. A sensitive ELISPOT assay for detection of CD8+ T lymphocytes specific for HLA class I-binding peptide epitopes derived from influenza proteins in the blood of healthy donors and melanoma patients. Clin. Cancer Res., 3: 221-226, 1997.

40. thor Straten, P., Guldberg, P., Grønbaek, K., Zeuthen, J., and Becker, J. C. In Situ T-Cell Responses against Melanoma Comprise High Numbers of Locally Expanded T-Cell Clonotypes. J. Immunol., 163: 443-447, 1999.

41. Kessler, J. H., Beekman, N. J., Bres-Vloemans, S. A., Verdijk, P., van Veelen, P. A., Kloosterman-Joosten, A. M., Vissers, D. C., ten Bosch, G. J., Kester, M. G., Sijts, A., Wouter, D. J., Ossendorp, F., Offringa, R., and Melief, C. J. Efficient identification of novel HLA-A(*)0201-presented cytotoxic T lymphocyte epitopes in the widely expressed tumor antigen PRAME by proteasome-mediated digestion analysis. J. Exp. Med., 193: 73-88, 2001.

42. de Vries, T. J., Fourkour, A., Wobbes, T., Verkroost, G., Ruiter, D. J., and van Muijen, G. N. Heterogeneous expression of immunotherapy candidate proteins gp100, MART-1, and tyrosinase in human melanoma cell lines and in human melanocytic lesions. Cancer Res., 57: 3223-3229, 1997.

43. Jager, E., Ringhoffer, M., Karbach, J., Arand, M., Oesch, F., and Knuth, A. Inverse relationship of melanocyte differentiation antigen expression in melanoma tissues and CD8+ cytotoxic-T-cell responses: evidence for immunoselection of antigen-loss variants in vivo. Int. J. Cancer, 66: 470-476, 1996.

44. Cormier, J. N., Abati, A., Fetsch, P., Hijazi, Y. M., Rosenberg, S. A., Marincola, F. M., and Topalian, S. L. Comparative analysis of the in vivo expression of tyrosinase, MART-1/Melan-A, and gp100 in metastatic melanoma lesions: implications for immunotherapy. J. Immunother., 21: 27-31, 1998.

45. Riker, A., Cornier, I., Panelli, M., Kammula, U., Wang, E., Abati, A., Fetsch, P., Lee, K. H., Steinberg, S., Rosenberg, S., and Marincola, F. Immune selection after antigen-specific immunotherapy of melanoma. Surgery, 126: 112-120, 1999.

46. Maeurer, M. J., Gollin, S. M., Martin, D., Swaney, W., Bryant, J., Castelli, C., Robbins, P., Parmiani, G., Storkus, W. J., and Lotze, M. T. Tumor escape from immune recognition: lethal recurrent melanoma in a patient associated with downregulation of the peptide transporter protein TAP-1 and loss of expression of the immunodominant MART-1/Melan-A antigen. J. Clin. Invest, 98: 1633-1641, 1996.

47. Grossman, D., Kim, P. J., Schechner, J. S., and Altieri, D. C. Inhibition of melanoma tumor growth in vivo by survivin targeting. Proc. Natl. Acad. Sci. U.S.A, 98: 635-640, 2001.

48. Tamm, I., Wang, Y., Sausville, E., Scudiero, D. A., Vigna, N., Oltersdorf, T., and Reed, J. C. IAP-family protein survivin inhibits caspase activity and apoptosis induced by Fas (CD95), Bax, caspases, and anticancer drugs. Cancer Res., 58: 5315-5320, 1998.

49. Monzo, M., Rosell, R., Felip, E., Astudillo, J., Sanchez, J. J., Maestre, J., Martin, C., Font, A., Barnadas, A., and Abad, A. A novel anti-apoptosis gene: Re-expression of survivin messenger RNA as a prognosis marker in non-small-cell lung cancers. J. Clin. Oncol., 17: 2100-2104, 1999.

50. Nakagawara, A. Molecular basis of spontaneous regression of neuroblastoma: role of neurotrophic signals and genetic abnormalities. Hum. Cell, 11: 115-124, 1998.

51. Renkvist, N., Castelli, C., Robbins, P. F., and Parmiani, G. A listing of human tumor antigens recognized by T cells. Cancer Immunol Immunother., 50: 3-15, 2001.

52. Melief, C. J., Toes, R. E., Medema, J. P., van der Burg, S. H., Ossendorp, F., and Offringa, R. Strategies for immunotherapy of cancer. Adv. Immunol., 75:235-82.: 235-282, 2000.

53. Gilboa, E. The makings of a tumor rejection antigen. Immunity, 11: 263-270, 1999.

54. Li, F., Ambrosini, G., Chu, E. Y., Plescia, J., Tognin, S., Marchisio, P. C., and Altieri, D. C. Control of apoptosis and mitotic spindle checkpoint by survivin. Nature, 396: 580-584, 1998.

55. Zaffaroni, N. and Daidone, M. G. Survivin expression and resistance to anticancer treatments: perspectives for new therapeutic interventions. Drug Resist. Updat., 5: 65-72, 2002.

56. Shinozawa, I., Inokuchi, K., Wakabayashi, I., and Dan, K. Disturbed expression of the anti-apoptosis gene, survivin, and EPR-1 in hematological malignancies. Leuk. Res, 24: 965-970, 2000.

57. Granziero, L., Ghia, P., Circosta, P., Gottardi, D., Strola, G., Geuna, M., Montagna, L., Piccoli, P., Chilosi, M., and Caligaris-Cappio, F. Survivin is expressed on CD40 stimulation and interfaces proliferation and apoptosis in B-cell chronic lymphocytic leukemia. Blood, 97: 2777-2783, 2001.

58. Ambrosini, G., Adida, C., and Altieri, D. C. A novel anti-apoptosis gene, survivin, expressed in cancer and lymphoma. Nat. Med., 3: 917-921, 1997.

59. Altieri, D. C. Validating survivin as a cancer therapeutic target. Nat. Rev. Cancer, 3: 46-54, 2003.

60. Olie, R. A., Simoes-Wust, A. P., Baumann, B., Leech, S. H., Fabbro, D., Stahel, R. A., and Zangemeister-Wittke, U. A novel antisense oligonucleotide targeting survivin expression induces apoptosis and sensitizes lung cancer cells to chemotherapy. Cancer Res, 60: 2805-2809, 2000.

61. Andersen, M. H. and thor Straten, P. Survivin—a universal tumor antigen. Histol. Histopathol., 17: 669-675, 2002.

62. Andersen, M. H., Pedersen, L. O., Capeller, B., Brocker, E. B., Becker, J. C., and thor, S. P. Spontaneous cytotoxic T-cell responses against survivin-derived MHC class I-restricted T-cell epitopes in situ as well as ex vivo in cancer patients. Cancer Res, 61: 5964-5968, 2001.

63. Currier, J. R., Kuta, E. G., Turk, E., Earhart, L. B., Loomis-Price, L., Janetzki, S., Ferrar, G., Birx, D. L., and Cox, J. H. A panel of MHC class I restricted viral peptides for use as a quality control for vaccine trial ELISPOT assays. J. Immunol. Methods, 260: 157-172, 2002.

64. Elvin, J., Potter, C., Elliott, T., Cerundolo, V., and Townsend, A. A method to quantify binding of unlabeled peptides to class I MHC molecules and detect their allele specificity. J Immunol Methods, 158: 161-171, 1993.

65. Ruppert, J., Sidney, J., Celis, E., Kubo, R. T., Grey, H. M., and Sette, A. Prominent role of secondary anchor residues in peptide binding to HLA-A2.1 molecules. Cell, 74: 929-937, 1993.

66. thor Straten, P., Barfoed, A., Seremet, T., Saeterdal, I., Zeuthen, J., and Guldberg, P. Detection and characterization of alpha-beta-T-cell clonality by denaturing gradient gel electrophoresis (DGGE). Biotechniques, 25: 244-250, 1998.

67. Rammensee, H., Bachmann, J., Emmerich, N. P., Bachor, O. A., and Stevanovic, S. SYFPEITHI: database for MHC ligands and peptide motifs. Immunogenetics, 50: 213-219, 1999.

68. Schrama, D., Pedersen Ls, L. O., Keikavoussi, P., Andersen, M. H., Straten, P. P., Brocker, E. B., Kampgen, E., and Becker, J. C. Aggregation of antigen-specific T cells at the inoculation site of mature dendritic cells. J. Invest Dermatol., 119: 1443-1448, 2002.

69. Mahotka, C., Wenzel, M., Springer, E., Gabbert, H. E., and Gerharz, C. D. Survivin-deltaEx3 and survivin-2B: two novel splice variants of the apoptosis inhibitor survivin with different antiapoptotic properties. Cancer Res., 59: 6097-6102, 1999.

70. Hicklin, D. J., Marincola, F. M., and Ferrone, S., HLA class I antigen downregulation in human cancers: T-cell immunotherapy revives an old story. Mol. Med. Today, 5: 178-186, 1999.

71. Seliger, B., Cabrera, T., Garrido, F., and Ferrone, S. HLA class I antigen abnormalities and immune escape by malignant cells. Semin. Cancer Biol., 12: 3-13, 2002.

72. Sette, A., Vitiello, A., Reherman, B., Fowler, P., Nayersina, R., Kast, W. M., Melief, C. J., Oseroff, C., Yuan, L., Ruppert, J., and . The relationship between class I binding affinity and immunogenicity of potential cytotoxic T cell epitopes. J. Immunol., 153: 5586-5592, 1994.

73. Moudgil, K. D. and Sercarz, E. E. Can antitumor immune responses discriminate between self and nonself? Immunol. Today, 15: 353-355, 1994.

74. Parkhurst, M. R., Salgaller, M. L., Southwood, S., Robbins, P. F., Sette, A., Rosenberg, S. A., and Kawakami, Y. Improved induction of melanoma-reactive CTL with peptides from the melanoma antigen gp100 modified at HLA-A*0201-binding residues. J. Immunol., 157: 2539-2548, 1996.

75. Guichard, G., Zerbib, A., Le Gal, F. A., Hoebeke, J., Connan, F., Choppin, J., Briand, J. P., and Guillet, J. G. Melanoma peptide MART-1(27-35) analogues with enhanced binding capacity to the human class I histocompatibility molecule HLA-A2 by introduction of a beta-amino acid residue: implications for recognition by tumor-infiltrating lymphocytes. J. Med. Chem., 43: 3803-3808, 2000.

76. Clay, T. M., Custer, M. C., McKee, M. D., Parkhurst, M., Robbins, P. F., Kerstann, K., Wunderlich, J., Rosenberg, S. A., and Nishimura, M. I. Changes in the fine specificity of gp100(209-217)-reactive T cells in patients following vaccination with a peptide modified at an HLA-A2.1 anchor residue. J. Immunol., 162: 1749-1755, 1999.

77. Melief, C. J., van der Burg, S. H., Toes, R. E., Ossendorp, F., and Offringa, R. Effective therapeutic anticancer vaccines based on precision guiding of cytolytic T lymphocytes. Immunol. Rev., 188: 177-182, 2002.

78. Jager, E., Ringhoffer, M., Altmannsberger, M., Arand, M., Karbach, J., Jager, D., Oesch, F., and Knuth, A. Immunoselection in vivo: independent loss of MHC class I and melanocyte differentiation antigen expression in metastatic melanoma. Int. J. Cancer, 71: 142-147, 1997.

79. Thurner, B., Haendle, I., Roder, C., Dieckmann, D., Keikavoussi, P., Jonuleit, H., Bender, A., Maczek, C., Schreiner, D., von den, D. P., Brocker, E. B., Steinman, R. M., Enk, A., Kämpgen, E., and Schuler, G. Vaccination with mage-3A1 peptide-pulsed mature, monocyte-derived dendritic cells expands specific cytotoxic T cells and induces regression of some metastases in advanced stage IV melanoma. J. Exp. Med., 190: 1669-1678, 1999.

80. Yee, C., Thompson, J. A., Roche, P., Byrd, D. R., Lee, P. P., Piepkorn, M., Kenyon, K., Davis, M. M., Riddell, S. R., and Greenberg, P. D. Melanocyte destruction after antigen-specific immunotherapy of melanoma: direct evidence of t cell-mediated vitiligo. J. Exp. Med., 192: 1637-1644, 2000.

81. Simon, R. M., Steinberg, S. M., Hamilton, M., Hildesheim, A., Khleif, S., Kwak, L. W., Mackall, C. L., Schlom, J., Topalian, S. L., and Berzofsky, J. A. Clinical trial designs for the early clinical development of therapeutic cancer vaccines. J. Clin. Oncol., 19: 1848-1854, 2001.

1. A MHC Class I-restricted epitope peptide derived from survivin, said epitope having at least one of the following characteristics:
(i) capable of binding to the Class I HLA molecule to which it is restricted at an affinity as measured by the amount of the peptide that is capable of half maximal recovery of the Class I HLA molecule (C₅₀ value) which is at the most 50 μM as determined by the assembly binding assay as described herein,
(ii) capable of eliciting INF-γ-producing cells in a PBL population of a cancer patient at a frequency of at least 1 per 10⁴ PBLs as determined by an ELISPOT assay, and/or
(iii) capable of in situ detection in a tumor tissue of CTLs that are reactive with the epitope peptide.

2. A peptide according to claim 1 having a C₅₀ value, which is at the most 30 μM.

3. A peptide according to claim 2 having a C₅₀ value, which is at the most 20 μM.

4. A peptide according to claim 1, which is restricted by a MHC Class I HLA-A molecule.

5. A peptide according to claim 4, which is restricted by a MHC Class I HLA species selected from the group consisting of HLA-A1, HLA-A2, HLA-A3, HLA-A11 and HLA-A24.

6. A peptide according to claim 5, which is restricted by HLA-A2.

7. A peptide according to claim 6, which is selected from the group consisting of FLKLDRERA (SEQ ID NO:1), TLPPAWQPFL (SEQ ID NO:2), ELTLGEFLKL (SEQ ID NO:3), LLLGEFLKL (SEQ ID NO:4) and LMLGEFLKL (SEQ ID NOno:5).

8. A peptide according to claim 1, which is restricted by a MHC Class I HLA-B molecule.

9. A peptide according to claim 8, which is restricted by a MHC Class I HLA-B species selected from the group consisting of HLA-B7, HLA -B35, HLA -B44, HLA-B8, HLA-B15, HLA-B27 and HLA-B51.

10. A peptide according to claim 9, which is restricted by HLA-B35.

11. A peptide according to claim 10, which is selected from the group consisting of CPTENEPDL (SEQ ID NO:6), EPDLAQCFF (SEQ ID NO:7), CPTENEPDY (SEQ ID NO:8) and EPDLAQCFY (SEQ ID NO:9).

12. A peptide according to claim 1 comprising at the most 20 amino acid residues.

13. A peptide according to claim 12 that comprises at the most 10 amino acid residues.

14. A peptide according to claim 1, which is a nonapeptide or a decapeptide.

15. A peptide according to claim 1, which is a native sequence of survivin of a mammal species.

16. A peptide according to claim 15 that is derived from human survivin.

17. A peptide according to claim 1, which is derived from a native sequence of survivin by substituting, deleting or adding at least one amino acid residue.

18. A peptide according to claim 1 comprising, for each specific HLA allele, any of the amino acid residues as indicated in the following table:

| HLA allele | Position 1 | Position 2 | Position 3 | Position 5 | Position 6 | Position 7 | C-terminal |
|---|---|---|---|---|---|---|---|
| HLA-A1 | | T, S | D, E | | | L | Y |
| HLA-A2 | | L, M | | | V | | L, V |
| HLA-A3 | | L, V, M | F, Y | | | | K, Y, F |
| HLA-A11 | | V, I, F, Y | M, L, F, Y, I | | | | K, R |
| HLA-A23 | | I, Y | | | | | W, I |
| HLA-A24 | | Y | | I, V | F | | I, L, F |
| HLA-A25 | | M, A, T | I | | | | W |
| HLA-A26 | E, D | V, T, I, L, F | | | I, L, V | | Y, F |
| HLA-A28 | E, D | V, A, L | | | | | A, R |
| HLA-A29 | | E | | | | | Y, L |
| HLA-A30 | | Y, L, F, V | | | | | Y |
| HLA-A31 | | | L, M, F, Y | | | | R |
| HLA-A32 | | I, L | | | | | W |
| HLA-A33 | | Y, I, L, V | | | | | R |
| HLA-A34 | | V, L | | | | | R |
| HLA-A66 | E, D | T, V | | | | | R, K |
| HLA-A68 | E, D | T, V | | | | | R, K |
| HLA-A69 | | V, T, A | | | | | V, L |
| HLA-A74 | | T | | | | | V, L |
| HLA-B5 | | A, P | F, Y | | | | I, L |
| HLA-B7 | | P | | | | | L, F |
| HLA-B8 | | | K | K, R | | | L |
| HLA-B14 | | R, K | | | | | L, V |
| HLA-B15 | | Q, L, K, P, | | | | | F, Y, W |
| (B62) | | H, V, I, M, | | | | | |
| | | S, T | | | | | |
| HLA-B17 | | | | | | | L, V |
| HLA-B27 | | R | | | | | Y, K, F, L |
| HLA-B35 | | P | | | | | I, L, M, Y |
| HLA-B37 | | D, E | | | | | I, L, M |
| HLA-B38 | | H | D, E | | | | F, L |
| HLA-B39 | | R, H | | | | | L, F |
| HLA-B40 | | E | F, I, V | | | | L, V, A, W, |
| (B60, 61) | | | | | | | M, T, R |
| HLA-B42 | | L, P | | | | | Y, L |
| HLA-B44 | | E | | | | | F, Y, W |
| HLA-B46 | | M, I, L, V | | | | | Y, F |
| HLA-B48 | | Q, K | | | | | L |
| HLA-B51 | | A, P, G | | | | | F, Y, I, V |
| HLA-B52 | | Q | F, Y | | | | I, V |
| HLA-B53 | | P | | | | | W, F, L |
| HLA-B54 | | P | | | | | |
| HLA-B55 | | P | | | | | A, V |
| HLA-B56 | | P | | | | | A, V |
| HLA-B57 | | A, T, S | | | | | F, W, Y |
| HLA-B58 | | A, T, S | | | | | F, W, Y |
| HLA-B67 | | P | | | | | L |
| HLA-B73 | | R | | | | | P |
| HLA- | | A, L | | | | | L |
| Cw1 | | | | | | | |
| HLA- | | A, L | | | | | F, Y |
| Cw2 | | | | | | | |
| HLA- | | A, L | | | | | L, M |
| Cw3 | | | | | | | |
| HLA- | | Y, P, F | | | | | L, M, F, Y |
| Cw4 | | | | | | | |
| HLA- | | | | | | | L, I, V, Y |
| Cw6 | | | | | | | |
| HLA- | | Y | | | | | L, Y, F |
| Cw6 | | | | | | | |
| HLA- | | Y | | | | | L, I, |
| Cw8 | | | | | | | |
| HLA- | | A, L | | | | | L, V |
| Cw16 | | | | | | | |

19. A peptide according to claims 1 that is capable of eliciting INF-γ-producing cells in a PBL population of a cancer patient at a frequency of at least 10 per 10⁴ PBLs.

20. A peptide according to claim 1, which is capable of eliciting INF-γ-producing cells in a PBL population of a patient having a cancer disease where survivin is expressed.

21. A peptide according to claim 20 where the cancer disease is selected from the group consisting of a haematopoietic malignancy including chronic lymphatic leukemia and chronic myeloid leukemia, melanoma, breast cancer, cervix cancer, ovary cancer, lung cancer, colon cancer, pancreas cancer and prostate cancer.

22. A peptide according to claim 1, which is capable of eliciting INF-γ-producing cells in a PBL population of a patient having a cancer disease, said INF-γ-producing cells having cytotoxic effect against survivin expressing cells of a cancer cell line, including a cell line selected from the group consisting of the breast cancer cell line MCF-7 and the melanoma cell line FM3.

23. A pharmaceutical composition comprising the peptide according to claim 1.

24. A composition according to claim 23 that comprises a peptide according to claim 4 in combination with a peptide according to claim 8.

25. A composition according to claim 24 comprising a peptide according to claim 6 in combination with a peptide according to claim 10.

26. A composition according to claim 23, which is a vaccine capable of eliciting an immune response against a cancer disease.

27. A composition according to claim 26 where the vaccine is capable of eliciting an immune response against a cancer disease where survivin is expressed.

28. A composition according to claim 27 where the cancer disease is selected from the group consisting of a haematopoietic malignancy, melanoma, breast cancer, cervix cancer, ovary cancer, lung cancer, colon cancer, pancreas cancer and prostate cancer.

29. A composition according to claim 27 or 28 where the vaccine elicits the production in the vaccinated subject of effector T-cells having a cytotoxic effect against the cancer cells.

30. A composition for ex vivo or in situ diagnosis of the presence in a cancer patient of survivin reactive T-cells among PBLs or in tumor tissue, the composition comprising a peptide according to claim 1.

31. A diagnostic kit for ex vivo or in situ diagnosis of the presence in a cancer patient of survivin reactive T-cells among PBLs or in tumour tissue comprising a peptide according to claim 1.

32. A complex of a peptide according to claims 1 and a Class I HLA molecule or a fragment of such molecule.

33. A complex according to claim 32 which is monomeric.

34. A complex according to claim 32 which is multimeric.

35. A method of detecting in a cancer patient the presence of survivin reactive T-cells, the method comprising contacting a tumour tissue or a blood sample with a complex according to claim 31 and detecting binding of the complex to the tissue or the blood cells.

36. A molecule that is capable of binding specifically to a peptide according to claim 1.

37. A molecule according to claim 36 which is an antibody or a fragment hereof.

38. A molecule that is capable of blocking the binding of a molecule according to claim 36 or 37.

39. A method of treating a cancer disease, the method comprising administering to a patient suffering from the disease an effective amount of the composition according to claim 23, a molecule according to claim 36 or a molecule according to claim 38.

40. A method according to claim 39 wherein the disease to be treated is a cancer disease where survivin is expressed.

41. A method according to claim 40 wherein the cancer disease is selected from the group consisting of a haematopoietic malignancy, melanoma, breast cancer, cervix cancer, ovary cancer, lung cancer, colon cancer, pancreas cancer and prostate cancer.

42. A method according to claim 39, which is combined with a further treatment.

43. A method according to claim 42 wherein the further treatment is radiotherapy or chemotherapy.

44. A composition according to claim 28 wherein the haematopoietic malignancy is chronic lymphatic leukemia or chronic myeloid leukemia.

45. A method according to claim 41 wherein the haematopoietic malignancy is chronic lymphatic leukemia or chronic myeloid leukemia.


SEQUENCE LISTING (65 sequences)

SEQ ID NO: 1 - protein, 9 aa, Artificial Sequence
  Sur6 peptide
FLKLDRERA

SEQ ID NO: 2 - protein, 10 aa, Artificial Sequence
  Sur8 peptide
TLPPAWQPFL

SEQ ID NO: 3 - protein, 10 aa, Artificial Sequence
  Sur9 peptide
ELTLGEFLKL

SEQ ID NO: 4 - protein, 9 aa, Artificial Sequence
  Sur1L2 peptide
LLLGEFLKL

SEQ ID NO: 5 - protein, 9 aa, Artificial Sequence
  Sur1M2 peptide
LMLGEFLKL

SEQ ID NO: 6 - protein, 9 aa, Artificial Sequence
  Sur 46-54 peptide
CPTENEPDL

SEQ ID NO: 7 - protein, 9 aa, Artificial Sequence
  Sur51-59 peptide
EPDLAQCFF

SEQ ID NO: 8 - protein, 9 aa, Artificial Sequence
  Sur46Y9 peptide
CPTENEPDY

SEQ ID NO: 9 - protein, 9 aa, Artificial Sequence
  sur51Y9 peptide
EPDLAQCFY

SEQ ID NO: 10 - protein, 9 aa, Artificial Sequence
  Sur1 peptide
LTLGEFLKL

SEQ ID NO: 11 - protein, 9 aa, Artificial Sequence
  C1 peptide
ILKEPVHGV

SEQ ID NO: 12 - protein, 9 aa, Artificial Sequence
  Sur2 peptide
RAIEQLAAM

SEQ ID NO: 13 - protein, 9 aa, Artificial Sequence
  Sur3 peptide
KVRRAIEQL

SEQ ID NO: 14 - protein, 9 aa, Artificial Sequence
  Sur4 peptide
STFKNWPFL

SEQ ID NO: 15 - protein, 9 aa, Artificial Sequence
  Sur5 peptide
SVKKQFEEL

SEQ ID NO: 16 - protein, 9 aa, Artificial Sequence
  Sur7 peptide
TAKKVRRAI

SEQ ID NO: 17 - protein, 10 aa, Artificial Sequence
  Sur10 peptide
ETAKKVRRAI

SEQ ID NO: 18 - protein, 9 aa, Artificial Sequence
  Sur 6-14 peptide
LPPAWQPFL

SEQ ID NO: 19 - protein, 9 aa, Artificial Sequence
  Sur 11-19 peptide
QPFLKDHRI

SEQ ID NO: 20 - protein, 10 aa, Artificial Sequence
  Sur 34-43 peptide
TPERMAEAGF

SEQ ID NO: 21 - protein, 9 aa, Artificial Sequence
  C24 peptide
YPLHEQHQM

SEQ ID NO: 22 - protein, 9 aa, Artificial Sequence
  Sur14-22 peptide
LKDHRISTF

SEQ ID NO: 23 - protein, 9 aa, Artificial Sequence
  Sur38-46 peptide
MAEAGFIHC

SEQ ID NO: 24 - protein, 9 aa, Artificial Sequence
  Sur93-101  peptide
FEELTLGEF

SEQ ID NO: 25 - protein, 10 aa, Artificial Sequence
  Sur47-56 peptide
PTENEPDLAQ

SEQ ID NO: 26 - protein, 10 aa, Artificial Sequence
  Sur49-58 peptide
ENEPDLAQCF

SEQ ID NO: 27 - protein, 10 aa, Artificial Sequence
  Sur92-101 peptide
QFEELTLGEF

SEQ ID NO: 28 - protein, 9 aa, Artificial Sequence
  C1 peptide
VSDGGPNLY

SEQ ID NO: 29 - protein, 9 aa, Artificial Sequence
  sur14Y9 peptide
LKDHRISTY

SEQ ID NO: 30 - protein, 9 aa, Artificial Sequence
  sur93Y9 peptide
FEELTLGEY

SEQ ID NO: 31 - protein, 10 aa, Artificial Sequence
  sur92Y9 peptide
QFEELTLGEY

SEQ ID NO: 32 - protein, 10 aa, Artificial Sequence
  sur34Y9 peptide
TPERMAEAGY

SEQ ID NO: 33 - protein, 10 aa, Artificial Sequence
  sur49Y9 peptide
ENEPDLAQCY

SEQ ID NO: 34 - protein, 10 aa, Artificial Sequence
  Sur92T2 peptide
QTEELTLGEF

SEQ ID NO: 35 - protein, 10 aa, Artificial Sequence
  Sur92S2 peptide
QSEELTLGEF

SEQ ID NO: 36 - protein, 9 aa, Artificial Sequence
  Sur93T2 peptide
FTELTLGEF

SEQ ID NO: 37 - protein, 9 aa, Artificial Sequence
  Sur93S2 peptide
FSELTLGEF

SEQ ID NO: 38 - protein, 9 aa, Artificial Sequence
  Sur38Y9 peptide
MAEAGFIHY

SEQ ID NO: 39 - protein, 10 aa, Artificial Sequence
  Sur46Y10 peptide
PTENEPDLAY

SEQ ID NO: 40 - protein, 9 aa, Artificial Sequence
  Sur 5-13 peptide
TLPPAWQPF

SEQ ID NO: 41 - protein, 9 aa, Artificial Sequence
  Sur 53-61 peptide
DLAQCFFCF

SEQ ID NO: 42 - protein, 9 aa, Artificial Sequence
  Sur 54-62 peptide
LAQCFFCFK

SEQ ID NO: 43 - protein, 9 aa, Artificial Sequence
  Sur 95-103 peptide
ELTLGEFLK

SEQ ID NO: 44 - protein, 9 aa, Artificial Sequence
  Sur 112-120 peptide
KIAKETNNK

SEQ ID NO: 45 - protein, 10 aa, Artificial Sequence
  Sur 13-22 peptide
FLKDHRISTF

SEQ ID NO: 46 - protein, 10 aa, Artificial Sequence
  Sur 18-26 peptide
RISTFKNWPF

SEQ ID NO: 47 - protein, 10 aa, Artificial Sequence
  Sur 53-62 peptide
DLAQCFFCFK

SEQ ID NO: 48 - protein, 10 aa, Artificial Sequence
  Sur 84-92 peptide
CAFLSVKKQF

SEQ ID NO: 49 - protein, 10 aa, Artificial Sequence
  Sur 101-120 peptide
FLKLDRERAK

SEQ ID NO: 50 - protein, 10 aa, Artificial Sequence
  Sur 103-112 peptide
KLDRERAKNK

SEQ ID NO: 51 - protein, 10 aa, Artificial Sequence
  Sur 112-121 peptide
KIAKETNNKK

SEQ ID NO: 52 - protein, 10 aa, Artificial Sequence
  Sur 113-125 peptide
IAKETNNKKK

SEQ ID NO: 53 - protein, 9 aa, Artificial Sequence
  C3 peptide
ILRGSVAHK

SEQ ID NO: 54 - protein, 9 aa, Artificial Sequence
  Sur5K9 peptide
TLPPAWQPK

SEQ ID NO: 55 - protein, 9 aa, Artificial Sequence
  Sur53K9 peptide
DLAQCFFCK

SEQ ID NO: 56 - protein, 9 aa, Artificial Sequence
  Sur54L2 peptide
LLQCFFCFK

SEQ ID NO: 57 - protein, 10 aa, Artificial Sequence
  Sur13K9 peptide
FLKDHRISTK

SEQ ID NO: 58 - protein, 10 aa, Artificial Sequence
  Sur18K9 peptide
RISTFKNWPK

SEQ ID NO: 59 - protein, 10 aa, Artificial Sequence
  Sur113L2 peptide
ILKETNNKKK

SEQ ID NO: 60 - protein, 9 aa, Artificial Sequence
  SurEx3-A3-1 peptide
TIRRKNLRK

SEQ ID NO: 61 - protein, 10 aa, Artificial Sequence
  SurEx3-A3-2 peptide
PTIRRKNLRK

SEQ ID NO: 62 - protein, 9 aa, Artificial Sequence
  Sur2b-A3-1 peptide
RITREEHKK

SEQ ID NO: 63 - protein, 10 aa, Artificial Sequence
  C4 peptide
AVFDRKSDAK

SEQ ID NO: 64 - protein, 9 aa, Artificial Sequence
  C6 peptide
QPRAPIRPI

SEQ ID NO: 65 - protein, 9 aa, Artificial Sequence
  C7 peptide
RPPIFIRRL